(19) United States
(12) Patent Application Publication
(10) Pub. No.: US 2015/0225726 A1
(43) Pub. Date: Aug. 13, 2015

(54) MICROORGANISMS AND PROCESSES FOR THE CONVERSION OF GLYCEROL TO ISOPRENE

(71) Applicant: GLYCOS BIOTECHNOLOGIES, INC., Houston, TX (US)

(72) Inventors: Sebastian Bredow, Houston, TX (US); Stephanie Doneske, Katy, TX (US); Mai Li, Katy, TX (US); Huaijin Zhou, Houston, TX (US); Daniel J. Monticello, The Woodlands, TX (US); Paul Campbell, Houston, TX (US)

(21) Appl. No.: 14/598,485

(22) Filed: Jan. 16, 2015

Related U.S. Application Data
(63) Continuation of application No. PCT/US2013/051194, filed on Jul. 19, 2013.
(60) Provisional application No. 61/741,460, filed on Jul. 20, 2012.

(51) Int. Cl. C12N 15/52; C12P 5/00

(52) U.S. Cl. CPC C12N 15/52; C12P 5/007

(57) ABSTRACT

The present invention provides non-naturally occurring microorganisms that have been modified to produce isoprene from glycerol. The microorganisms include one of several glycerol dissimilation pathways and one of several isoprene production pathways.

### RELATED APPLICATION

This application is a continuation application of International Application No. PCT/US2013/051194, which designated the United States and was filed on Jul. 19, 2013, published in English, which claims the benefit of U.S. Provisional Application No. 61/741,460, filed on Jul. 20, 2012. The entire teachings of the above applications are incorporated herein by reference.

### FIELD OF THE INVENTION

The present disclosure generally relates to the use of a non-naturally occurring microorganism for the production of isoprene from glycerol. More specifically, the present disclosure relates to non-naturally occurring microorganisms that have been modified to overexpress genes of the glycerol dissimilation pathway, overexpress genes of the methylerythritol pathway or mevalonate pathway, and reduce or eliminate expression of genes that lead to the formation of unwanted co-products.

### BACKGROUND OF THE INVENTION

Currently, many high-value chemicals or fuels are typically manufactured by chemical synthesis from hydrocarbons, including petroleum oil and natural gas. Also, high value chemicals may be produced as “by-products” during the processing of crude oil into usable fractions. For example, isoprene has typically been produced during the catalytic cracking of crude oil. However, as catalytic crackers have shifted their focus from crude oil to natural gas, there is now a reduced source of the four and five carbon chain molecules that are found in crude oil, but not natural gas.

Being a short-chain carbon source, isoprene is a useful early or initial component for synthesizing a variety of chemicals. Isoprene may be used as a monomer or co-monomer. Examples of chemicals that can be produced using isoprene include polyisoprene, polybutylene, styrene-isoprene-styrene block co-polymers, and others. An example of an industry that uses isoprene is the synthetic rubber industry.

Given the demand for and the many uses of isoprene, a new method of isoprene production is desired. Also, as the concerns of energy security, increasing oil and natural gas prices, and global warming escalate, the chemical production industry is seeking ways to replace chemicals made from non-renewable feedstocks with chemicals produced from renewable feedstocks using environmentally friendly practices.

Glycerol is gaining prominence as a useful carbon source for large-scale fermentations. While glycerol may be produced from petrochemical feedstocks, it is also produced as a co-product of the oleochemical and biodiesel industries, generally through acid splitting or transesterification of oils and fats from animal or vegetable origin. With the growth of the oleochemical and biodiesel industries, there is now an abundance of glycerol available for use as a carbon source in fermentations.

While examples of the conversion of glycerol to isoprene via fermentation are known, these approaches suffer from low carbon flux from glycerol to the important metabolic intermediate, dimethylallyl diphosphate. Furthermore, co-products produced from the glycerol reduce the yield of final product, isoprene, while complicating separations and recovery steps.

Thus, there is a need and a desire for non-naturally occurring microorganisms for the efficient conversion of glycerol to isoprene.

### SUMMARY OF THE INVENTION

Embodiments of the present invention generally provide non-naturally occurring microorganisms that produce isoprene from glycerol via the methylerythritol pathway or mevalonate pathway and methods of producing isoprene from glycerol using said non-naturally occurring microorganisms.

In some aspects, the present invention improves the carbon flux from glycerol to dimethylallyl diphosphate, and further conversion from dimethylallyl diphosphate to isoprene, by (i) improving the rate at which glycerol is converted into the glycolytic intermediate dihydroxyacetone phosphate, (ii) improving the rate of conversion of glyceraldehyde-3-phosphate (G3P) and pyruvate (PYR) into dimethylallyl diphosphate or improving the rate of conversion of acetyl-CoA to dimethylallyl diphosphate, and (iii) reducing or eliminating the production of undesirable co-products from the glycerol, such as acetate, lactate, ethanol, and succinate.

In one embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, a 2-methyl-3-buten-2-ol synthase, and a 2-methyl-3-buten-2-ol dehydratase, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase, 2-methyl-3-buten-2-ol synthase, and 2-methyl-3-buten-2-ol dehydratase are encoded by an exogenous nucleic acid. In certain embodiments, each of the enzymes in the isoprene pathway listed above is encoded by exogenous nucleic acids. The glycerol dissimilation and isoprene pathways are expressed in sufficient quantities to produce isoprene from glycerol.

In another embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, a 3-methyl-2-buten-1-ol synthase, a 2-methyl-3-buten-2-ol isomerase, and a 2-methyl-3-buten-2-ol dehydratase, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase, 3-methyl-2-buten-1-ol synthase, 2-methyl-3-buten-2-ol isomerase, and 2-methyl-3-buten-2-ol dehydratase are encoded by an exogenous nucleic acid. In certain embodiments, each of the enzymes in the isoprene pathway listed above are encoded by exogenous nucleic acids. The glycerol dissimilation and isoprene pathways are expressed in sufficient quantities to produce isoprene from glycerol.

In another embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, and a monoterpene synthase, sesquiterpene synthase or diterpene synthase capable of converting dimethylallyl diphosphate into isoprene, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase and monoterpene synthase, sesquiterpene synthase or diterpene synthase are encoded by an exogenous nucleic acid. In certain embodiments, each of the enzymes in the isoprene pathway listed above are encoded by exogenous nucleic acids. The glycerol dissimilation and isoprene pathways are expressed in sufficient quantities to produce isoprene from glycerol.

In another embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, and an isoprene synthase, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase and isoprene synthase are encoded by an exogenous nucleic acid. In certain embodiments, each of the enzymes in the isoprene pathway listed above are encoded by exogenous nucleic acids. The glycerol dissimilation and isoprene pathways are expressed in sufficient quantities to produce isoprene from glycerol.

In another embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising an isoprene pathway comprising a 1-deoxyxylulose-5-phosphate synthase, a 1-deoxy-D-xylulose-5-phosphate reductoisomerase, a 4-diphosphocytidyl-2-C-methyl-D-erythritol synthase, a 4-diphosphocytidyl-2-C-methylerythritol kinase, a 1-hydroxy-2-methyl-2-(E)-butenyl-4-diphosphate synthase, a dimethylallyl-diphosphate/isopentenyl-diphosphate:NAD(P)′ oxidoreductase, an isopentenyl diphosphate isomerase, a 2-methyl-3-buten-2-ol synthase, and a 2-methyl-3-buten-2-ol dehydratase, wherein one or more of the 1-deoxyxylulose-5-phosphate synthase, 1-deoxy-D-xylulose-5-phosphate reductoisomerase, 4-diphosphocytidyl-2-C-methyl-D-erythritol synthase, 4-diphosphocytidyl-2-C-methylerythritol kinase, 1-hydroxy-2-methyl-2-(E)-butenyl-4-diphosphate synthase, dimethylallyl-diphosphate/isopentenyl-diphosphate:NAD(P)′ oxidoreductase, isopentenyl diphosphate isomerase, 2-methyl-3-buten-2-ol synthase, and 2-methyl-3-buten-2-ol dehydratase are encoded by an exogenous nucleic acid. In certain embodiments, each of the enzymes in the isoprene pathway listed above are encoded by exogenous nucleic acids. The glycerol dissimilation and isoprene pathways are expressed in sufficient quantities to produce isoprene from glycerol.

In another embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising a 1-deoxyxylulose-5-phosphate synthase, a 1-deoxy-D-xylulose-5-phosphate reductoisomerase, a 4-diphosphocytidyl-2-C-methyl-D-erythritol synthase, a 4-diphosphocytidyl-2-C-methylerythritol kinase, a 1-hydroxy-2-methyl-2-(E)-butenyl-4-diphosphate synthase, a dimethylallyl-diphosphate/isopentenyl-diphosphate:NAD(P)⁺ oxidoreductase, an isopentenyl diphosphate isomerase, a 3-methyl-2-buten-1-ol synthase, a 2-methyl-3-buten-2-ol isomerase, and a 2-methyl-3-buten-2-ol dehydratase, wherein one or more of the 1-deoxyxylulose-5-phosphate synthase, 1-deoxy-D-xylulose-5-phosphate reductoisomerase, 4-diphosphocytidyl-2-C-methyl-D-erythritol synthase, 4-diphosphocytidyl-2-C-methylerythritol kinase, 1-hydroxy-2-methyl-2-(E)-butenyl-4-diphosphate synthase, dimethylallyl-diphosphate/isopentenyl-diphosphate:NAD(P)′ oxidoreductase, isopentenyl diphosphate isomerase, 3-methyl-2-buten-1-ol synthase, 2-methyl-3-buten-2-ol isomerase and 2-methyl-3-buten-2-ol dehydratase are encoded by an exogenous nucleic acid. In certain embodiments, each of the enzymes in the isoprene pathway listed above are encoded by exogenous nucleic acids. The glycerol dissimilation and isoprene pathways are expressed in sufficient quantities to produce isoprene from glycerol.

In a further embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising a 1-deoxyxylulose-5-phosphate synthase, a 1-deoxy-D-xylulose-5-phosphate reductoisomerase, a 4-diphosphocytidyl-2-C-methyl-D-erythritol synthase, a 4-diphosphocytidyl-2-C-methylerythritol kinase, a 1-hydroxy-2-methyl-2-(E)-butenyl-4-diphosphate synthase, a dimethylallyl-diphosphate/isopentenyl-diphosphate:NAD(P)′ oxidoreductase, an isopentenyl diphosphate isomerase, and a monoterpene synthase, sesquiterpene synthase, or diterpene synthase capable of converting dimethylallyldiphosphate into isoprene, wherein one or more of the 1-deoxyxylulose-5-phosphate synthase, 1-deoxy-D-xylulose-5-phosphate reductoisomerase, 4-diphosphocytidyl-2-C-methyl-D-erythritol synthase, 4-diphosphocytidyl-2-C-methylerythritol kinase, 1-hydroxy-2-methyl-2-(E)-butenyl-4-diphosphate synthase, dimethylallyl-diphosphate/isopentenyl-diphosphate:NAD(P)′ oxidoreductase, isopentenyl diphosphate isomerase, and the monoterpene synthase, sesquiterpene synthase, or diterpene synthase are encoded by an exogenous nucleic acid. In certain embodiments, each of the enzymes in the isoprene pathway listed above are encoded by exogenous nucleic acids. The glycerol dissimilation and isoprene pathways are expressed in sufficient quantities to produce isoprene from glycerol.

In a further embodiment, the non-naturally occurring microorganism includes a glycerol dissimilation pathway that comprises either a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and the dihydroxyacetone kinase are encoded by an exogenous nucleic acid. In certain embodiments, each of the glycerol kinase and glycerol-3-phosphate dehydrogenase or the glycerol dehydrogenase and dihydroxyacetone kinase is encoded by exogenous nucleic acids. The microorganism also includes an isoprene pathway comprising a 1-deoxyxylulose-5-phosphate synthase, a 1-deoxy-D-xylulose-5-phosphate reductoisomerase, a 4-diphosphocytidyl-2-C-methyl-D-erythritol synthase, a 4-diphosphocytidyl-2-C-methylerythritol kinase, a 1-hydroxy-2-methyl-2-(E)-butenyl-4-diphosphate synthase, a dimethylallyl-diphosphate/isopentenyl-diphosphate:NAD(P)′ oxidoreductase, an isopentenyl diphosphate isomerase, and an isoprene synthase, wherein one or more of the 1-deoxyxylulose-5-phosphate synthase, 1-deoxy-D-xylulose-5-phosphate reductoisomerase, 4-diphosphocytidyl-2-C-methyl-D-erythritol synthase, 4-diphosphocytidyl-2-C-methylerythritol kinase, 1-hydroxy-2-methyl-2-(E)-butenyl-4-diphosphate synthase, dimethylallyl-diphosphate/isopentenyl-diphosphate:NAD(P)′ oxidoreductase, isopentenyl diphosphate isomerase, and isoprene synthase are encoded by an exogenous nucleic acid. In certain embodiments, each of the enzymes in the isoprene pathway listed above are encoded by exogenous nucleic acids. The glycerol dissimilation and isoprene pathways are expressed in sufficient quantities to produce isoprene from glycerol.

In one embodiment, the non-naturally occurring microorganism converts the dimethylallyl diphosphate produced by the methylerythritol pathway into isoprene using an isoprene synthase polypeptide.

In an additional embodiment, the non-naturally occurring microorganism converts the dimethylallyl diphosphate produced by the methylerythritol pathway into isoprene using a monoterpene synthase, sesquiterpene synthase, or diterpene synthase.

In one embodiment, the non-naturally occurring microorganism converts the dimethylallyl diphosphate produced by the methylerythritol pathway into 3-methyl-2-buten-1-ol by a 3-methyl-2-buten-1-ol synthase, the 3-methyl-2-buten-1-ol is converted to 2-methyl-3-buten-2-ol by a 2-methyl-3-buten-2-ol isomerase, and the 2-methyl-3-buten-2-ol is converted to isoprene by a 2-methyl-3-buten-2-ol dehydratase.

In another embodiment, the non-naturally occurring microorganism converts the dimethylallyl diphosphate produced by the methylerythritol pathway into 2-methyl-3-buten-2-ol by a methylbutenol synthase, and 2-methyl-3-buten-2-ol is converted to isoprene by a 2-methyl-3-buten-2-ol dehydratase.

In some aspects, the present invention improves the carbon flux from glycerol to dimethylallyl diphosphate, and further conversion from dimethylallyl diphosphate to isoprene, by (i) improving the rate at which glycerol is converted into the glycolytic intermediate dihydroxyacetone phosphate, (ii) improving the rate of conversion of acetyl-CoA into dimethylallyl diphosphate, and (iii) reducing or eliminating the production of undesirable co-products from the glycerol.

In an embodiment, the non-naturally occurring microorganism converts the dimethylallyl diphosphate produced by the mevalonate pathway into isoprene using an isoprene synthase polypeptide.

In an additional embodiment, the non-naturally occurring microorganism converts the dimethylallyl diphosphate produced by the mevalonate pathway into isoprene using a monoterpene synthase, sesquiterpene synthase, or diterpene synthase.

In one embodiment, the non-naturally occurring microorganism converts the dimethylallyl diphosphate produced by the mevalonate pathway into 3-methyl-2-buten-1-ol by a 3-methyl-2-buten-1-ol synthase, the 3-methyl-2-buten-1-ol is converted to 2-methyl-3-buten-2-ol by a 2-methyl-3-buten-2-ol isomerase, and the 2-methyl-3-buten-2-ol is converted to isoprene by a 2-methyl-3-buten-2-ol dehydratase.

In another embodiment, the non-naturally occurring microorganism converts the dimethylallyl diphosphate produced by the mevalonate pathway into 2-methyl-3-buten-2-ol by a methylbutenol synthase, and 2-methyl-3-buten-2-ol is converted to isoprene by a 2-methyl-3-buten-2-ol dehydratase.

Embodiments of the invention also provide methods of producing isoprene comprising culturing the microorganisms described herein under suitable conditions in a medium containing glycerol for a sufficient period of time to produce isoprene from the glycerol. The isoprene is then recovered.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows: A. A glycerol dissimilation pathway that uses glycerol kinase and either an aerobic glycerol-3-phosphate dehydrogenase (encoded by glpD in Escherichia coli) or an anaerobic glycerol-3-phosphate dehydrogenase (encoded by glpABC in Escherichia coli); and B. A glycerol dissimilation pathway that uses glycerol dehydrogenase (gldA) and either a phosphoenolpyruvate-dependent dihydroxyacetone kinase (dhaKLM) or an adenosine triphosphate-(ATP-) dependent dihydroxyacetone kinase (dhaKL).

FIG. 2 shows a methylerythritol phopshate metabolic pathway for the conversion of glyceraldehyde-3-phosphate and pyruvate to dimethylallyl diphosphate, comprising the enzymes: 1-deoxy-D-xylulose-5-phosphate synthase, 1-deoxy-D-xylulose-5-phosphate reductoisomerase, 4-diphosphocytidyl-2-C-methyl-D-erythritol synthase, 4-diphosphocytidyl-2-C-methyl-D-erythritol kinase, 2-C-methyl-D-erythritol-2,4-cyclodiphosphate synthase, 1-hydroxy-2-methyl-2-(E)-butenyl-4-diphosphate synthase, dimethylallyl-diphosphate/isopentenyl-diphosphate:NAD(P)⁺ oxidoreductase, and isopentenyl diphosphate isomerase.

FIG. 3 shows a metabolic pathway for the conversion of acetyl-CoA to dimethylallyl diphosphate, comprising the enzymes: acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, and isopentenyl diphosphate isomerase.

FIG. 4 shows: A. Conversion of dimethylallyl diphosphate to isoprene in a single enzymatic step; B. Conversion of dimethylallyl diphosphate into isoprene in a three-step enzymatic pathway comprising a 3-methyl-2-buten-1-ol synthase, a 2-methyl-3-buten-2-ol isomerase, and a 2-methyl-3-buten-2-ol dehydratase; and C. Conversion of dimethylallyl diphosphate into isoprene in a two-step enzymatic pathway comprising a 2-methyl-3-buten-2-ol isomerase, and a 2-methyl-3-buten-2-ol dehydratase.

FIG. 5 shows a gas chromatogram of a 1-milliliter sample of the headspace of a 20-milliliter vial containing E. coli strain BL21 harboring plasmid pJ404-LDI cultured overnight on 1 mM 3-methyl-2-buten-1-ol in Luria Bertani broth supplemented with 100 μg/ml ampicillin. Peak 1 is 3-methyl-2-buten-1-ol, with a retention time of 3.96 minutes. Peak 2 is 2-methyl-3-buten-2-ol, with a retention time of 2.96 minutes. Peak 3 is isoprene, with a retention time of 2.49 minutes.

FIG. 6 shows a gas chromatogram of a 1-milliliter sample of the headspace of a 20-milliliter vial containing E. coli strain BL21 harboring plasmid pJ404-LDI cultured overnight on 1 mM 2-methyl-3-buten-2-ol in Luria Bertani broth supplemented with 100 μg/ml ampicillin. Peak 1 is 2-methyl-3-buten-2-ol, with a retention time of 2.96 minutes. Peak 2 is isoprene, with a retention time of 2.49 minutes.

FIG. 7 shows a fermentation of glycerol to lactic acid using E. coli strain LA02 harboring plasmid pZS-adhEp.glpK.glpD.

FIG. 8 shows a codon-optimized nucleic acid sequence [SEQ ID NO: 33], including an artificial ribosome binding site and an amino-terminal 6-histidine epitope tag, for the linalool dehydratase-isomerase of Castellaniella defragrans strain 65Phen.

FIG. 9 shows a codon-optimized nucleic acid sequence [SEQ ID NO: 34], including an artificial ribosome binding site and an amino-terminal 6-histidine epitope tag, for strawberry alcohol acyltransferase.

FIG. 10 shows a gas chromatogram of a 1-milliliter sample of the headspace of a 20-milliliter vial containing 1 mM 3-methyl-2-buten-1-ol in Luria Bertani broth. Peak 1 is 3-methyl-2-buten-1-ol, with a retention time of 3.96 minutes.

FIG. 11 shows the results of GC/MS analysis of authentic isoprene.

FIG. 12 shows the results of GC/MS analysis of the peak at 2.49 minutes from Example 1, verifying the identity of the peak as isoprene.

FIG. 13 shows a gas chromatogram of a 1-milliliter sample of the headspace of a 20-milliliter vial containing 1 mM 2-methyl-3-buten-2-ol in Luria Bertani broth. Peak 1 is 2-methyl-3-buten-2-ol, with a retention time of 2.96 minutes.

FIG. 14 shows the DNA sequence [SEQ ID NO: 35] of plasmid pGA31R-mcs.

FIG. 15 shows the DNA sequence [SEQ ID NO: 36] of plasmid pGE21R-mcs.

FIG. 16 shows the codon-optimized sequence [SEQ ID NO: 37] of the mvaE and mvaS genes of Enterococcus faecalis ATCC 700802, including incorporated ribosome binding sites and flanking restriction endonuclease sites used in subsequent cloning steps.

FIG. 17 shows the codon-optimized sequence [SEQ ID NO: 38] of the synthetic operon encoding the mevalonate kinase gene of Methanocaldococcus jannaschi, the phosphomevalonate kinase gene of Enterococcus faecalis ATCC 700802, the mevalonate pyrophosphate decarboxylase gene of Saccharomyces cerevisiae S288C, and the isopentenyl diphosphate isomerase gene of E. coli MG1655, including incorporated ribosome binding sites and flanking restriction endonuclease sites used in subsequent cloning steps.

FIG. 18 shows the codon-optimized sequence [SEQ ID NO: 39] of the synthetic isoprene synthase gene of Populus alba, without the encoded N-terminal chloroplast transit peptide but containing a ribosome binding site and flanking restriction endonuclease sites used in subsequent cloning steps.

FIG. 19 shows a cloning strategy for the production of plasmids pGB 1004 and pGB1012.

FIG. 20 shows a cloning strategy for the production of plasmid pGB 1008 and pGB1012.

FIG. 21 shows a cloning strategy for the production of plasmid pGB1030.

FIG. 22 shows the codon-optimized sequence [SEQ ID NO: 40] of the 3-methyl-2-buten-1-ol synthase gene of Pinus sabiniana and the isopentenyl diphosphate isomerase gene of Haematococcus pluvialis, including ribosome binding sites and flanking restriction endonuclease sites used in subsequent cloning steps.

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within a glycerol dissimilation or isoprene biosynthetic pathway.

A metabolic modification refers to a biochemical reaction that is altered from its naturally occurring state. Therefore, non-naturally occurring microorganisms can have genetic modifications to nucleic acids encoding metabolic polypeptides or, functional fragments thereof. Exemplary metabolic modifications are disclosed herein.

As used herein, the term “isolated” when used in reference to a microbial organism is intended to mean an organism that is substantially free of at least one component as the referenced microbial organism is found in nature. The term includes a microbial organism that is removed from some or all components as it is found in its natural environment. The term also includes a microbial organism that is removed from some or all components as the microbial organism is found in non-naturally occurring environments. Therefore, an isolated microbial organism is partly or completely separated from other substances as it is found in nature or as it is grown, stored or subsisted in non-naturally occurring environments. Specific examples of isolated microbial organisms include partially pure microbes, substantially pure microbes and microbes cultured in a medium that is non-naturally occurring.

As used herein, the terms “microbe,” “microbial,” “microbial organism” or “microorganism” is intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

The non-naturally occurring microbial organisms of the invention can contain stable genetic alterations, which refer to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as Escherichia coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microorganism. An example of orthologs exhibiting separable activities is where distinct activities have been separated into distinct gene products between two or more species or within a single species.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms of the invention having glycerol dissimilation and/or isoprene biosynthetic capability, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microorganism that encode an enzyme catalyzing a similar or substantially similar metabolic reaction, those skilled in the art also can utilize these evolutionally related genes. Orthologs, paralogs and nonorthologous gene displacements can be determined by methods well known to those skilled in the art.

As defined herein, a knocked-out gene is a gene whose encoded product, e.g., a protein or polypeptide, does not or substantially does not perform its usual function or any function. A knocked-out gene can be created through deletion, disruption, insertion, or mutation. As defined herein, microorganisms that lack one or more indicated knocked-out genes are also considered to have knock outs of the indicated gene(s). The microorganisms themselves may also be referred to as knock outs of the indicated gene(s). Such knock outs can also be conditional or inducible, using techniques that are well-known to those of skill in the art. Also contemplated are “knock ins”, in which a gene, or one or more segments of a gene, are introduced into the microorganism in place of, or in addition to, the endogenous copy of the gene. Once again, many techniques for creating knock in microorganisms are known to those of ordinary skill in the art.

As defined herein, nucleic acids or enzymes described herein as being “from” or “derived from” certain organisms include codon-optimized versions of those nucleic acids or enzymes.

The methods and techniques utilized for culturing or generating the microorganisms disclosed herein are known to the skilled worker trained in microbiological and recombinant DNA techniques. Methods and techniques for growing microorganisms (e.g., bacterial cells), transporting isolated DNA molecules into the host cell and isolating, cloning and sequencing isolated nucleic acid molecules, knocking out expression of specific genes, etc., are examples of such techniques and methods. These methods are described in many items of the standard literature, which are incorporated herein in their entirety: “Basic Methods In Molecular Biology” (Davis, et al., eds. McGraw-Hill Professional, Columbus, Ohio, 1986); Miller, “Experiments in Molecular Genetics” (Cold Spring Harbor Laboratory Press, Cold Spring Harbor, N.Y., 1972); Miller, “A Short Course in Bacterial Genetics” (Cold Spring Harbor Laboratory Press, Cold Spring Harbor, N.Y., 1992); Singer and Berg, “Genes and Genomes” (University Science Books, Mill Valley, Calif., 1991); “Molecular Cloning: A Laboratory Manual,” 2^{nd} Ed. (Sambrook, et al., eds., Cold Spring Harbor Laboratory Press, Cold Spring Harbor, N.Y., 1989); “Handbook of Molecular and Cellular Methods in Biology and Medicine” (Kaufman, et al., eds., CRC Press, Boca Raton, Fla., 1995); “Methods in Plant Molecular Biology and Biotechnology” (Glick and Thompson, eds., CRC Press, Boca Raton, Fla., 1993); and Smith-Keary, “Molecular Genetics of Escherichia coli” (The Guilford Press, New York, N.Y., 1989).

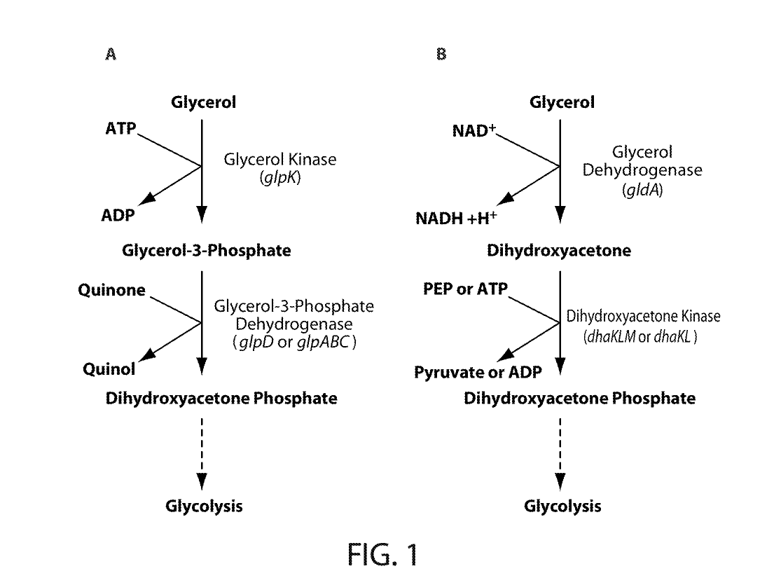

Embodiments of the present invention provide non-naturally occurring microorganisms for the production of isoprene having improved rates of glycerol dissimilation, improved rates of conversion of glyceraldehyde-3-phosphate and pyruvate to dimethylallyl diphosphate (also referred to as dimethylallyl pyrophosphate or DMAPP), and reduced amounts of co-products such as acetate, lactate, ethanol, and succinate. As shown in FIG. 1, glycerol can be converted to glycerol-3-phosphate by glycerol kinase, and from glycerol phosphate to dihydroxyacetone phosphate (DHAP) by glycerol-3-phosphate dehydrogenase. Alternatively, in a second glycerol dissimilation pathway, glycerol can be converted to dihydroxyacetone by glycerol dehydrogenase, and from dihydroxyacetone to dihydroxyacetone phosphate by a dihydroxyacetone kinase. In both pathways, the dihydroxyacetone phosphate then enters glycolysis to be converted to glyceraldehyde-3-phosphate and pyruvate. A methylerythritol phosphate pathway then converts the glyceraldehyde-3-phosphate and pyruvate to dimethylallyl diphosphate. A series of one or more steps then converts the dimethylallyl diphosphate into isoprene or another suitable isoprenoid molecule.

Additional embodiments of the present invention provide non-naturally occurring microorganisms for the production of isoprene having improved rates of glycerol dissimilation, improved rates of conversion of acetyl-CoA to dimethylallyl diphosphate, and reduced amounts of co-products such as acetate, lactate, ethanol, and succinate. As shown in FIG. 1, glycerol can be converted to glycerol-3-phosphate by glycerol kinase, and from glycerol phosphate to dihydroxyacetone phosphate (DHAP) by glycerol-3-phosphate dehydrogenase. Alternatively, in a second glycerol dissimilation pathway, glycerol can be converted to dihydroxyacetone by glycerol dehydrogenase, and from dihydroxyacetone to dihydroxyacetone phosphate by a dihydroxyacetone kinase. In both pathways, the dihydroxyacetone phosphate then enters glycolysis to be converted to pyruvate, followed by conversion to acetyl-CoA. A mevalonate pathway then converts the acetyl-CoA to dimethylallyl diphosphate. A series of one or more steps then converts the dimethylallyl diphosphate into isoprene or another suitable isoprenoid molecule.

The rate of glycerol dissimilation may be increased through overexpression of genes encoding a glycerol dissimilation pathway. An example of a glycerol dissimilation pathway includes the enzymes glycerol kinase and glycerol-3-phosphate dehydrogenase (FIG. 1 A).

Glycerol kinase (E.C. No. 2.7.1.30) converts glycerol to glycerol-3-phosphate. This enzyme activity is encoded by the E. coli gene glpK, Table 1, below. Other comparable enzyme activities and the corresponding genes are known in other organisms. For example, Mus musculus, Saccharomyces cerevisiae, Klebsiella pneumoniae and Bacillus subtilis all possess glycerol kinase activities. Additional examples of glycerol kinase enzymes are presented in Table 1.

TABLE 1

| | GenBank Ac- | |
|---|---|---|
| Locus | cession No. | Organism |
| GLPK_ECOLI | P0A6F3 | Escherichia coli K-12 |
| GLPK_KLEP7 | A6TFR2 | Klebsiella pneumoniae |
| | | (ATCC 700721) |
| GLPK_BACSU | P18157 | Bacillus subtilis |
| GLPK_MOUSE | Q64516 | Mus musculus |
| GLPK_YEAST | P32190 | Saccharomyces cerevisiae |

Glycerol-3-phosphate dehydrogenase (E.C. Nos. 1.1.1.8, 1.1.1.94, and 1.1.5.3) converts glycerol-3-phosphate to dihydroxyacetone phosphate with the concomitant reduction of an NAD(P)+ molecule to NAD(P)H or a quinone to a quinol. Glycerol-3-phosphate dehydrogenase enzymes belonging to all three E.C. classes are known. The Saccharomyces cerevisiae genes GPD1 and GPD2 encode glycerol-3-phosphate dehydrogenases of E.C. No. 1.1.1.8. The Candida versatilis gene CvGPDJ encodes a glycerol-3-phosphate dehydrogenase of E.C. No. 1.1.1.94. The E. coli gene glpD encodes a quinone-dependent glycerol-3-phosphate dehydrogenase of E.C. No. 1.1.5.3. The E. coli enzyme G1pD is generally active under aerobic conditions. The E. coli operon g1pABC also encodes a quinone-dependent glycerol-3-phosphate dehydrogenase of E.C. No. 1.1.5.3, but this G1pABC enzyme is generally active under anaerobic conditions. Additional examples of glycerol-3-phosphate dehydrogenase enzymes are presented in Table 2, below.

TABLE 2

| | GenBank Ac- | |
|---|---|---|
| Locus | cession No. | Organism |
| E.C. No. 1.1.1.8 | | |
| GPD1L_MOUSE | Q3ULJ0 | Mus musculus |
| GPDA_ARATH | Q9SCX9 | Arabidopsis thaliana |
| GPD1_SCHPO | P21696 | Schizosaccharomyces pombe |
| GPD2_ SCHPO | Q09845 | Schizosaccharomyces pombe |
| GPD1_YEAST | Q00055 | Saccharomyces cerevisiae |
| GPD2_YEAST | P41911 | Saccharomyces cerevisiae |
| E.C. No. 1.1.1.94 | | |
| GPDA1_LACDA | Q93FD0 | Lactobacillus delbrueckii |
| | | subsp. bulgaricus |
| GPDA_LACLA | Q9CFX6 | Lactococcus lactis subsp. Lactis |
| GPDA_RHIE6 | B3PQA3 | Rhizobium etli (strain CIAT 652) |
| GPDA_AGRT5 | Q8UC48 | Agrobacterium tumefaciens |
| | | (strain C58) |
| E. C. No. 1.1.5.3 | | |
| GLPA_ECOLI | P0A9C0 | Escherichia coli |
| GLPB_ECOLI | P13033 | Escherichia coli |
| GLPC_ECOLI | P0A996 | Escherichia coli |
| GLPD_ECOLI | P13035 | Escherichia coli |
| GLPD_BACSU | P18158 | Bacillus subtilis |

Another example of a glycerol dissimilation pathway includes the enzymes glycerol dehydrogenase and dihydroxyacetone kinase (FIG. 1.B.).

Glycerol dehydrogenase (E.C. No. 1.1.1.6) converts glycerol to dihydroxyacetone with the concomitant reduction of an NAD(P)⁺ molecule to NAD(P)H or a quinone to a quinol.

The E. coli gene gldA encodes a glycerol dehydrogenase of E.C. No. 1.1.1.6. Similar enzymes are known from Klebsiella pneumoniae and Citrobacter freundii. Additional examples of glycerol dehydrogenase enzymes are presented in Table 3, below.

TABLE 3

| | GenBank Ac- | |
|---|---|---|
| Locus | cession No. | Organism |
| GLDA_ECOLI | P0A9S5 | Escherichia coli K-12 |
| B2ZPN8_KLEPN | B2ZPN8 | Klebsiella pneumoniae |
| C4IIN3_CLOBU | C4IIN3 | Clostridium butyricum E4 |
| | | str. BoNT E BL5262 |
| GLDA_CITFR | P45511 | Citrobacter freundii |
| GLDA_PSEPU | P50173 | Pseudomonas putida |

Dihydroxyacetone kinase (E.C. No. 2.7.1.29) converts dihydroxyacetone to dihydroxyacetone phosphate. The E. coli operon dhaKLM encodes a dihydroxyacetone kinase that uses phosphoenolpyruvate as the phosphate donor, resulting in the conversion of dihydroxyacetone and phosphoenolpyruvate (PEP) to dihydroxyacetone phosphate and pyruvate. The Citrobacter freundii operon dhaKL encodes a dihydroxyacetone kinase that uses adenosine-5′-triphosphate (ATP) as the phosphate donor, resulting in the conversion of dihydroxyacetone and ATP to dihydroxyacetone phosphate and adenosine-5′-diphosphate (ADP). Additional examples of glycerol dehydrogenase enzymes are presented in Table 4, below.

TABLE 4

| | GenBank Ac- | |
|---|---|---|
| Locus | cession No. | Organism |
| DHAK_ECOLI | P76015 | Escherichia coli K-12 |
| DHAL_ECOLI | P76014 | Escherichia coli K-12 |
| DHAM_ECOLI | P37349 | Escherichia coli K-12 |
| DHAK_CITFR | P45510 | Citrobacter freundii |
| Q1KLC3_CITFR | Q1KLC3 | Citrobacter freundii |
| DAK1_YEAST | P54838 | Saccharomyces cerevisiae |

In some embodiments, a non-naturally occurring microbial organism of the invention is generated from a host that inherently contains the enzymatic capability to convert glycerol to dihydroxyacetone phosphate; however, increased rates of conversion of glycerol to dihydroxyacetone phosphate are desirable. Increased synthesis of dihydroxyacetone phosphate from glycerol can be achieved by, for example, overexpression of nucleic acids encoding one or more of the above-described glycerol dissimilation pathway enzymes or proteins. Overexpression of the glycerol dissimilation pathway enzyme or enzymes can be achieved by, for example, the exogenous expression of the endogenous gene or genes, or the exogenous expression of the heterologous gene or genes of a glycerol dissimilation pathway. An expression vector can be constructed to express one or more endogenous or heterologous genes of the glycerol dissimilation pathway, as exemplified herein, operably linked to expression control sequences functional in the host organism. Expression vectors applicable for use in the microbial host organisms of the invention include, for example, plasmids, phage vectors, viral vectors, episomes and artificial chromosomes, including vectors and selection sequences or markers operable for stable integration into a host chromosome. When two or more exogenous encoding nucleic acids are to be co-expressed, both nucleic acids can be inserted, for example, into a single expression vector or in separate expression vectors. In addition, a non-naturally occurring organism can be generated by mutagenesis of an endogenous gene that results in an increase in activity of an enzyme in the glycerol dissimilation pathway.

Glycerol crosses the microbial cell membrane and enters the cytoplasm through a process of facilitated diffusion. For example, the G1 pF polypeptide of E. coli, encoded by the gene glpF (Table 3, below), is a transmembrane channel protein that facilitates the entry of glycerol into the E. coli cell. glpF mutants have impaired growth on low concentrations of glycerol (Richey, D. P., and E. C. C. Lin. 1972. Importance of facilitated diffusion for effective utilization of glycerol by Escherichia coli. J. Bact. 112: 784-790). Overexpression of a glycerol facilitator may lead to improved rates of glycerol dissimilation. Examples of glycerol facilitator proteins are listed in Table 5, below.

TABLE 5

| | GenBank Ac- | |
|---|---|---|
| Locus | cession No. | Organism |
| NP_418362 | NP_418362 | Escherichia coli K-12 |
| AAO37928 | AAO37928 | Borrelia hermsii DAH |
| CAH19327 | CAH19327 | Yersinia pseudotuberculosis IP 32953 |
| BAB61772 | BAB61772 | Thermus aquaticus |
| AAO57623 | AAO57623 | Pseudomonas syringae pv. |
| | | tomato str. DC3000 |
| CAY72431 | CAY72431 | Erwinia pyrifoliae DSM 12163 |

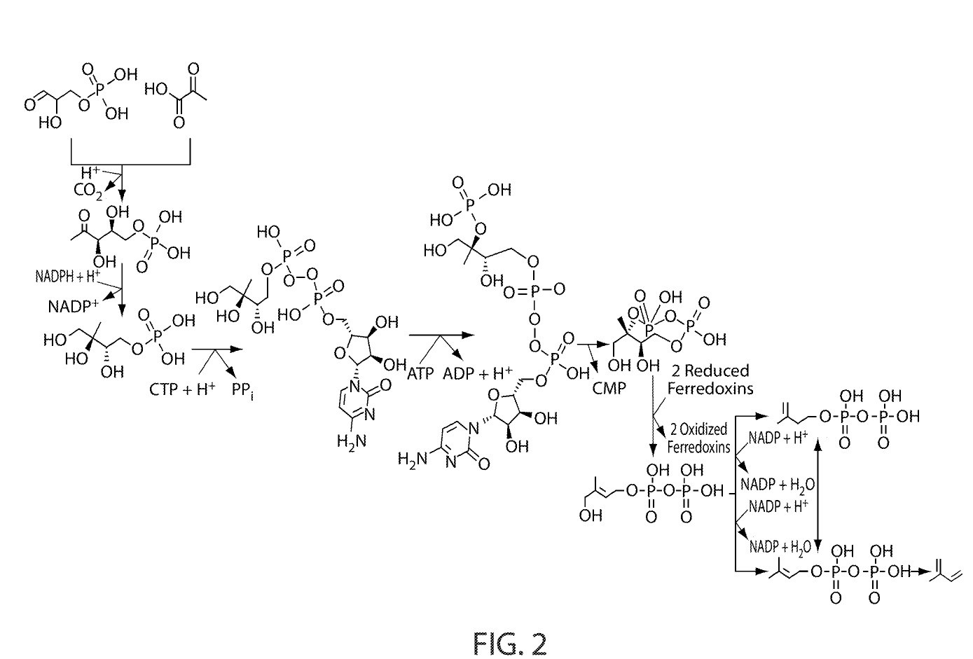

The rate of dimethylallyl diphosphate formation from glyceraldehyde-3-phosphate and pyruvate may be increased through overexpression of one or more genes encoding enzymes of the methylerythritol pathway (FIG. 2): 1-deoxy-D-xylulose-5-phosphate synthase, 1-deoxy-D-xylulose-5-phosphate reductoisomerase, 4-diphosphocytidyl-2-C-methyl-D-erythritol synthase, 4-diphosphocytidyl-2-C-methyl-D-erythritol kinase, 2-C-methyl-D-erythritol-2,4-cyclodiphosphate synthase, 1-hydroxy-2-methyl-2-(E)-butenyl-4-diphosphate synthase, dimethylallyl-diphosphate/isopentenyl-diphosphate:NAD(P)⁺ oxidoreductase, or isopentenyl diphosphate isomerase.

1-deoxy-D-xylulose-5-phosphate synthase (E.C. No. 2.2.1.7) converts glyceraldehyde-3-phosphate and pyruvate to 1-deoxy-D-xylulose-5-phosphate and carbon dioxide. This enzyme activity is encoded by the E. coli gene dxs. Other comparable enzyme activities and the corresponding genes are known in other organisms. Additional examples of 1-deoxy-D-xylulose-5-phosphate synthase enzymes are presented in Table 6, below.

TABLE 6

| | GenBank Ac- | |
|---|---|---|
| Locus | cession No. | Organism |
| DXS_ECOLI | P77488 | Escherichia coli K-12 |
| DXS_BACSU | P54523 | Bacillus subtilis |
| DXS_MYCTU | P0A554 | Mycobacterium tuberculosis |
| B4YSH1_SALMI | B4YSH1 | Salvia miltiorrhiza |
| DXS_HAEIN | P45205 | Haemophilu influenzae str. |
| | | ATCC 51907 |

1-deoxy-D-xylulose-5-phosphate reductoisomerase (E.C. No. 1.1.1.267) converts 1-deoxy-D-xylulose-5-phosphate to 2-C-methyl-D-erythritol-4-phosphate with the concomitant oxidation of NAD(P)H and H⁺ to NAD(P)⁺. This enzyme activity is encoded by the E. coli gene dxr. Other comparable enzyme activities and the corresponding genes are known in other organisms. Additional examples of 1-deoxy-D-xylulose-5-phosphate reductoisomerase enzymes are presented in Table 7, below.

TABLE 7

| | GenBank Ac- | |
|---|---|---|
| Locus | cession No. | Organism |
| DXR_ECOLI | P45568 | Escherichia coli K-12 |
| DXR_ BACSU | O31753 | Bacillus subtilis |
| DXR_MYCTU | P64012 | Mycobacterium tuberculosis |
| O96693_PLAFA | O96693 | Plasmodium falciparum |
| Q5J7B3_GINBI | Q5J7B3 | Ginkgo biloba |

4-diphosphocytidyl-2-C-methyl-D-erythritol synthase (E.C. No. 2.7.7.60) converts 2-C-methyl-D-erythritol-4-phosphate and cytidine-5′-triphosphate (CTP) to 4-diphosphocytidyl-2-C-methyl-D-erythritol with the release of inorganic pyrophosphate. This enzyme activity is encoded by the E. coli gene ispD. Other comparable enzyme activities and the corresponding genes are known in other organisms. Additional examples of 4-diphosphocytidyl-2-C-methyl-D-erythritol synthase enzymes are presented in Table 8, below.

TABLE 8

| | GenBank Ac- | |
|---|---|---|
| Locus | cession No. | Organism |
| ISPD_ECOLI | Q46893 | Escherichia coli K-12 |
| ISPD_BACSU | Q06755 | Bacillus subtilis |
| ISPD_MYCTU | P96864 | Mycobacterium tuberculosis |
| A9ZN10_HEVBR | A9ZN10 | Hevea brasiliensis |
| ISPD_ARATH | P69834 | Arabidopsis thaliana |

4-diphosphocytidyl-2-C-methyl-D-erythritol kinase (E.C. No. 2.7.1.148) converts 4-diphosphocytidyl-2-C-methyl-D-erythritol and ATP to 2-phospho-4-diphosphocytidyl-2-C-methyl-D-erythritol and ADP. This enzyme activity is encoded by the E. coli gene ispE. Other comparable enzyme activities and the corresponding genes are known in other organisms. Additional examples of 4-diphosphocytidyl-2-C-methyl-D-erythritol kinase enzymes are presented in Table 9, below.

TABLE 9

| | GenBank Ac- | |
|---|---|---|
| Locus | cession No. | Organism |
| ISPE_ECOLI | P62615 | Escherichia coli K-12 |
| ISPE_BACSU | P37550 | Bacillus subtilis |
| ISPETHET2 | Q72GN2 | Thermus thermophiles str. |
| | | ATCC BAA-163 |
| A6XAA8_GINBI | A6XAA8 | Ginkgo biloba |
| A6XAA9_BINBI | A6XAA9 | Ginkgo biloba |
| ISPE_ARATH | O81014 | Arabidopsis thaliana |

2-C-methyl-D-erythritol-2,4-cyclodiphosphate synthase (E.C. No. 4.6.1.12) converts 2-phospho-4-diphosphocytidyl-2-C-methyl-D-erythritol to 2-C-methyl-D-erythrito1-2,4-cyclodiphosphate and cytidine-5′-monophosphate (CMP). This enzyme activity is encoded by the E. coli gene ispF. Other comparable enzyme activities and the corresponding genes are known in other organisms. Additional examples of 2-C-methyl-D-erythrito1-2,4-cyclodiphosphate synthase enzymes are presented in Table 10, below.

TABLE 10

| | GenBank Ac- | |
|---|---|---|
| Locus | cession No. | Organism |
| ISPF_ECOLI | P62617 | Escherichia coli K-12 |
| ISPF_BACSU | Q06756 | Bacillus subtilis |
| ISPF_ENTFA | Q839V8 | Enterococcus faecalis |
| A9ZN13_HEVBR | A9ZN13 | Hevea brasiliensis |
| A1KXW3_HEVBR | A1KXW3 | Hevea brasiliensis |

1-hydroxy-2-methyl-2-(E)-butenyl-4-diphosphate synthase (E.C. No. 1.17.7.1) converts one molecule of 2-C-methyl-D-erythritol-2,4-cyclodiphosphate and two reduced ferredoxins to 1-hydroxy-2-methyl-2-(E)-butenyl-4-diphosphate and two oxidized ferredoxins with the concomitant release of one water molecule. This enzyme activity is encoded by the E. coli gene ispG. Other comparable enzyme activities and the corresponding genes are known in other organisms. Additional examples of 1-hydroxy-2-methyl-2-(E)-butenyl-4-diphosphate synthase enzymes are presented in Table 11, below.

TABLE 11

| | GenBank Ac- | |
|---|---|---|
| Locus | cession No. | Organism |
| ISPG_ECOLI | P62620 | Escherichia coli K-12 |
| ISPG_BACSU | P54482 | Bacillus subtilis |
| ISPG_THEEB | Q8DK70 | Thermosynechococcus elongatus |
| | | str. BP-1 |
| ISPG_CLOAB | Q97156 | Clostridium acetobutylicum |
| ISPG_AGRT5 | P58665 | Agrobacterium tumefaciens str. C58 |

Dimethylallyl-diphosphate/isopentenyl-diphosphate:NAD(P)⁺ oxidoreductase (E.C. No. 1.17.1.2) converts 1-hydroxy-2-methyl-2-(E)-butenyl-4-diphosphate into isopentenyl diphosphate and dimethylallyl diphosphate. This enzyme activity is encoded by the E. coli gene ispH, where the enzyme catalyzes the conversion of 1-hydroxy-2-methyl-2-(E)-butenyl-4-diphosphate into isopentenyl diphosphate and dimethylallyl diphosphate in a ratio of approximately 5 or 6 to 1 (for the E. coli enzyme; Rohdich, F., Hecht, S., Gartner, K., Adam, P., Kreiger, C., Amslinger, S., Arigoni, D., Bacher, A. and W. Eisenreich. 2002. Studies on the nonmevalonate terpene biosynthetic pathway: metabolic role of IspH (LytB) protein. Proc. Natl. Acad. Sci. USA 99: 1158-1163). In other organisms, where other comparable enzyme activities and the corresponding genes are known, the comparable enzyme may produce isopentenyl diphosphate and dimethylallyl diphosphate in different ratios. Additional examples of dimethylallyl-diphosphate/isopentenyl-diphosphate:NAD(P)⁺ oxidoreductase enzymes are presented in Table 12, below.

TABLE 12

| | GenBank Ac- | |
|---|---|---|
| Locus | cession No. | Organism |
| ISPH_ECOLI | P62623 | Escherichia coli K-12 |
| ISPH_BACSU | P54473 | Bacillus subtilis |
| ISPH_ZYMMO | Q5NP61 | Zymomonas mobilis |
| ISPH_AQUAE | O67625 | Aquifex aeolicus |
| A7AME9_BABBO | A7AME9 | Babesia bovis |

Isopentenyl diphosphate isomerase (E.C. No. 5.3.3.2) isomerizes isopentenyl diphosphate to dimethylallyl diphosphate in a reversible reaction. This enzyme activity is encoded by the E. coli gene idi. Other comparable enzyme activities and the corresponding genes are known in other organisms. Additional examples of isopentenyl diphosphate isomerase enzymes are presented in Table 13, below.

TABLE 13

| | GenBank Ac- | |
|---|---|---|
| Locus | cession No. | Organism |
| IDI_ECOLI | Q46822 | Escherichia coli K-12 |
| IDI2_BACSU | P50740 | Bacillus subtilis |
| IDI2_STRC1 | Q9KWG2 | Streptomyces sp. str. CL190 |
| O81660_HAEPL | O81660 | Haematococcus pluvialis |
| IDI1_YEAST | P15496 | Saccharomyces cerevisiae |
| IDI2_STAA9 | A5IVC7 | Staphylococcus aureus str. JH9 |
| IDI2_STAAM | P65102 | Staphylococcus aureus |
| | | ATCC 700699 |

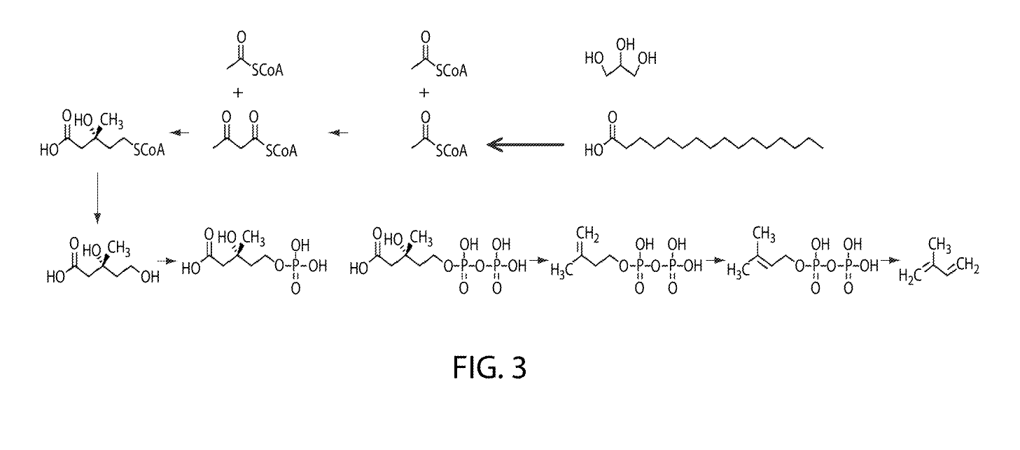

The rate of dimethylallyl diphosphate formation from acetyl-CoA may be increased through overexpression of one or more genes encoding enzymes of the mevalonate pathway (FIG. 3): acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, or isopentenyl diphosphate isomerase.

Acetyl-CoA C-acetyltransferase (E.C. No. 2.3.1.9) converts two molecules of acetyl-CoA to acetoacetyl-CoA and CoASH. This enzyme activity is encoded by the E. coli gene atoB. Other comparable enzyme activities and the corresponding genes are known in other organisms. Additional examples of acetyl-CoA C-acetyltransferase are presented in Table 14, below.

TABLE 14

| | GenBank Ac- | |
|---|---|---|
| Locus | cession No. | Organism |
| E.C. No. 2.3.1.9 | | |
| ATOB_ECOLI | P76461 | Escherichia coli K-12 |
| THLA_STAAM | Q99WM3 | Staphylococcus aureus |
| | | (ATCC 700699) |
| AF290094 | AF290094 | Enterococcus faecalis |
| Q8NN21_CORGL | Q8NN21 | Corynebacterium glutamicum |
| THL_BACSU | P45855 | Bacillus subtilis |
| THIC_MOUSE | Q8CAY6 | Mus musculus |
| THIC_MOUSE | Q8CAY6 | Mus musculus |
| QCR1_YEAST | P07256 | Saccharomyces cerevisiae |
| THLA_CLOAB | P45359 | Clostridium acetobutylicum |
| THIC_HUMAN | Q9BWD1 | Homo sapiens |

Hydroxymethylglutaryl-CoA synthase (E.C. No. 2.3.3.10) converts one molecule of acetyl-CoA and one molecule of acetoacetyl-CoA to (S)-3-hydroxy-3-methylglutaryl-CoA and CoASH. This enzyme is encoded by the Saccharomyces cerevisiae locus HMCS_YEAST. Other comparable enzyme activities and the corresponding genes are known in other organisms. Additional examples of hydroxymethylglutaryl-CoA synthase are presented in Table 15, below.

TABLE 15

| | GenBank Ac- | |
|---|---|---|
| Locus | cession No. | Organism |
| E.C. No. 2.3.3.10 | | |
| HMCS1_MOUSE | Q8JZK9 | Mus musculus |
| Q9FD87_STAAU | Q9FD87 | Staphylococcus aureus |
| Q9FD71_ENTFA | Q9FD71 | Enterococcus faecalis |
| B8ZMJ0_STRPJ | B8ZMJ0 | Streptococcus pneumoniae |
| | | (strain ATCC 700669) |
| A9ZMZ6_HEVBR | A9ZMZ6 | Hevea brasiliensis |
| HMCS_YEAST | P54839 | Saccharomyces cerevisiae |

Hydroxymethylglutaryl-CoA reductase converts (S)-3-hydroxy-3-methylglutaryl-CoA to mevalonic acid with the concomitant oxidation of two molecules of NAD(P)H. This enzyme is encoded by the Saccharomyces cerevisiae locus HMDH1_YEAST. Other comparable enzyme activities and the corresponding genes are known in other organisms. Additional examples of hydroxymethylglutaryl-CoA reductase are presented in Table 16, below.

TABLE 16

| | GenBank Ac- | |
|---|---|---|
| Locus | cession No. | Organism |
| E.C. No. 1.1.1.34 | | |
| HMDH_METJA | Q58116 | Methanocaldococcus jannaschii |
| Y1225_METJA | Q58622 | Methanocaldococcus jannaschii |
| AF290094 | AF290094 | Enterococcus faecalis |
| HMDH1_HEVBR | P29057 | Hevea brasiliensis |
| HMDH1_ARATH | P14891 | Arabidopsis thaliana |
| A5IVX2_STAA9 | A5IVX2 | Staphylococcus aureus (strain JH9) |
| HMDH1_YEAST | P12683 | Saccharomyces cerevisiae |
| HMDH2_YEAST | P12684 | Saccharomyces cerevisiae |
| D1XUL0_9ACTO | D1XUL0 | Streptomyces sp. ACTE |
| HMDH_ARCFU | O28538 | Archaeoglobus fulgidus |
| D4GU18_HALVD | D4GU18 | Haloferax volcanii |
| | | (strain ATCC 29605) |
| HMDH_SULSO | O08424 | Sulfolobus solfataricus |
| M24015 | M24015 | Pseudomonas mevalonii |

In some instances, the acetyl-CoA C-acetyltransferase and hydroxymethylglutaryl-CoA reductase activities may be encoded by a single gene. One such example is the mvaE gene of Enterococcus faecalis (Hedl, M., Sutherlin, A., Wilding, E. I., Mazzulla, M., McDevitt, D., Lane, P., Burgner, J. W. II, Lehnbeuter, K. R., Stauffacher, C. V., Gwynn, M. N., and V. W. Rodwell, 2002. Enterococcus faecalis acetoacetyl-Coenzyme A thiolase/3-hydroxy-3-methylglutaryl-coenzyme A reductase, a dual-function protein of ispentenyl diphosphate biosynthesis. J. Bacteriol. 184: 2116-2122).

Mevalonate kinase (E.C. No. 2.7.1.36) converts mevalonic acid and ATP to 5-phosphomevalonate and ADP. This enzyme is encoded by the KIME_YEAST locus of Saccharomyces cerevisiae. Other comparable enzyme activities and the corresponding genes are known in other organisms. Additional examples of mevalonate kinase are presented in Table 17, below.

TABLE 17

| | GenBank Ac- | |
|---|---|---|
| Locus | cession No. | Organism |
| E.C. No. 2.7.1.36 | | |
| KIME_METJA | Q58487 | Methanocaldococcus jannaschii |
| D4V248_ENTFA | D4V248 | Enterococcus faecalis PC1.1 |
| KIME_ARATH | P46086 | Arabidopsis thaliana |
| A5IQE4_STAA9 | A5IQE4 | Staphylococcus aureus (strain JH9) |
| KIME_YEAST | P07277 | Saccharomyces cerevisiae |
| B8ZLF2_STRPJ | B8ZLF2 | Streptococcus pneumoniae |
| | | (strain ATCC 700669) |
| KIME_ARCFU | O27995 | Archaeoglobus fulgidus |
| D4GWT5_HALVD | D4GWT5 | Haloferax volcanii |
| | | (strain ATCC 29605) |
| Q980D2_SULSO | Q980D2 | Sulfolobus solfataricus |

Phosphomevalonate kinase (E.C. No. 2.7.4.2) converts 5-phosphomevalonate and ATP to 5-diphosphomevalonate and ADP. This enzyme is encoded by the ERG8_YEAST locus of Saccharomyces cerevisiae. Other comparable enzyme activities and the corresponding genes are known in other organisms. Additional examples of phosphomevalonate kinase are presented in Table 18, below.

TABLE 18

| | GenBank Ac- | |
|---|---|---|
| Locus | cession No. | Organism |
| E.C. No. 2.7.4.2 | | |
| D4V246_ENTFA | D4V246 | Enterococcus faecalis PC1.1 |
| A9ZN02_HEVBR | A9ZN02 | Hevea brasiliensis |
| Q2FJ51_STAA3 | Q2FJ51 | Staphylococcus aureus |
| | | (strain USA300) |
| ERG8_YEAST | P24521 | Saccharomyces cerevisiae |

Diphosphomevalonate decarboxylase (E.C. No. 4.1.1.33) converts 5-diphosphomevalonate and ATP to ADP, phosphate, isopentenyl diphosphate and carbon dioxide. This enzyme is encoded by the MVD1_YEAST locus of Saccharomyces cerevisiae. Other comparable enzyme activities and the corresponding genes are known in other organisms. Additional examples of diphosphomevalonate decarboxylase are presented in Table 19, below.

TABLE 19

| | GenBank Ac- | |
|---|---|---|
| Locus | cession No. | Organism |
| E.C. No. 4.1.1.33 | | |
| D4V247_ENTFA | D4V247 | Enterococcus faecalis PC1.1 |
| A9ZN03_HEVBR | A9ZN03 | Hevea brasiliensis |
| O23722_ARATH | O23722 | Arabidopsis thaliana |
| A5IQE5_STAA9 | A5IQE5 | Staphylococcus aureus (strain JH9) |
| MVD1_YEAST | P32377 | Saccharomyces cerevisiae |
| B8ZLF3_STRPJ | B8ZLF3 | Streptococcus pneumoniae |
| | | (strain ATCC 700669) |
| D0KQL8_SULS9 | D0KQL8 | Sulfolobus solfataricus |
| | | (strain 98/2) |

Isopentenyl diphosphate can be converted to dimethylallyl diphosphate by isopentenyl diphosphate isomerase (Table 13, above).

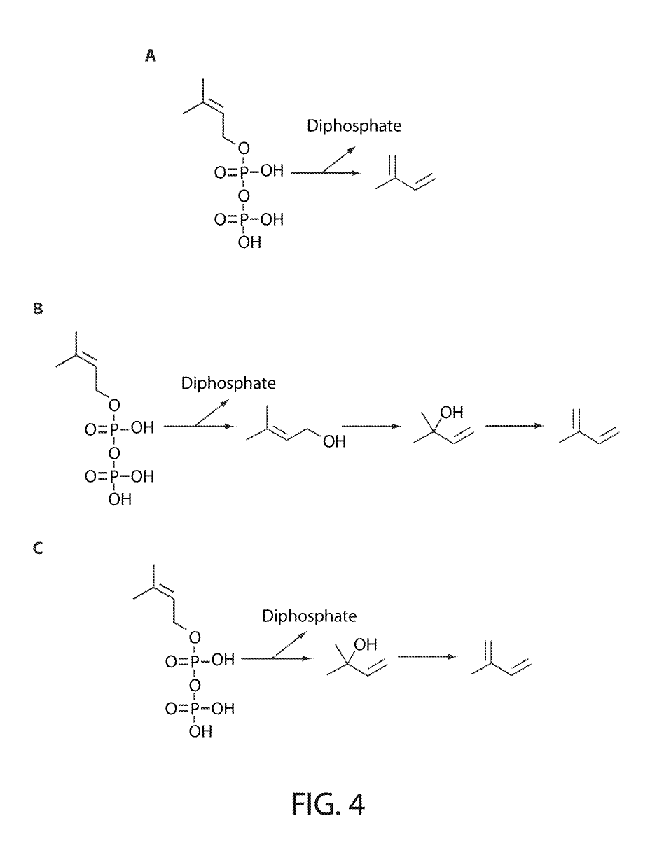

Dimethylallyl diphosphate may be converted to isoprene by several routes, including, but not limited to: 1) direct conversion of dimethylallyl diphosphate to isoprene by an isoprene synthase; 2) direct conversion of dimethylallyl diphosphate to isoprene by a mutant terpene synthase, for example a myrcene synthase or farnesene synthase, with substrate specificity altered from its natural substrate (geranyl diphosphate or farnesyl diphosphate, respectively) to dimethylallyl diphosphate; 3) a three-step isoprene biosynthetic pathway comprising the steps of direct conversion of dimethylallyl diphosphate to 3-methyl-2-buten-1-ol followed by enzymatic isomerization to 2-methyl-3-buten-2-ol and dehydration of 2-methyl-3-buten-2-ol to isoprene; or 4) a two-step isoprene biosynthetic pathway comprising the steps of direct conversion of dimethylallyl diphosphate to 2-methyl-3-buten-2-ol followed by dehydration of 2-methyl-3-buten-2-ol to isoprene. These routes are depicted in FIG. 4.

As used herein, isoprene synthases are nuclearly encoded, naturally occurring polypeptides found in some plant plastids, particularly in the chloroplast, that convert dimethylallyl diphosphate to isoprene, and derivatives (mutants) of polypeptides that naturally convert dimethylallyl diphosphate to isoprene (FIG. 4. A.). Isoprene synthases are characterized, in part, by an amino-terminal plastid targeting sequence (“transit peptide”) that routes the polypeptide to the chloroplast. Upon translocation into the chloroplast, the transit peptide may be cleaved from the polypeptide to yield a mature protein that is smaller in molecular weight than the precursor protein. Isoprene synthases have been found in several species of poplar tree (Populus spp.) and in kudzu (Pueraria montana var. lobata) with activities verified in vitro. Additional coding sequences predicted to encode isoprene synthases have been identified in other plant species. Exemplary isoprene synthase genes are presented in Table 20, below.

TABLE 20

| | GenBank Ac- | |
|---|---|---|
| Locus | cession No. | Organism |
| ISPS_POPCN | Q9AR86 | Populus canescens |
| ISPS_POPAL | Q50L36 | Populus alba |
| ISPS_PUEML | Q6EJ97 | Pueraria montana var. lobata |
| ISPS_POPTM | Q7XAS7 | Populus tremuloides |
| A0PFK2_POPNI | AOPFK2 | Populus nigra |
| A9Q7C9_POPAL | A9Q7C9 | Populus alba |
| D8UY75_POPAL | D8UY75 | Populus alba |
| D8UY76_POPAL | D8UY76 | Populus alba |
| H2CSU6_ROBPS | H2CSU6 | Robinia pseudoacacia |

For overexpression of an heterologous isoprene synthase in a microbial organism, it is preferable to express a truncated isoprene synthase that approximates the mature form found in nature, rather than the precursor form. Essentially, the sequence encoding the transit peptide is removed from the isoprene synthase coding sequence. While visual inspection may allow one skilled in the art to select where to truncate the isoprene synthase coding sequence, computer-based algorithms such as ChloroP 1.1 can be used to help predict which amino acids belong to the transit peptide (Emanuelsson, O., Nielsen, H., G. von Heijne. 1999. ChloroP, a neural network-based method for predicting chloroplast transit peptides and their cleavage sites. Protein Sci. 8: 978-984). H2CSU6 from Robinia pseudoacacia is predicted to be a truncated isoprene synthase coding region. It lacks an amino-terminal methionine and a proposed chloroplast transit peptide.

The direct conversion of dimethylallyl diphosphate to isoprene may be catalyzed by a mutant terpene synthase, for example a myrcene synthase or farnesene synthase, with substrate specificity altered from its natural substrate (geranyl diphosphate or farnesyl diphosphate, respectively) to dimethylallyl diphosphate (FIG. 4. A.). Table 21, below, provides examples of terpene synthases for use in the conversion of dimethylallyl diphosphate to isoprene.

TABLE 21

| | GenBank Ac- | |
|---|---|---|
| Locus | cession No. | Organism |
| Myrcene Synthase | | |
| (E.C. No. 4.2.3.15) | | |
| MYRS_OCIBA | Q5SBP1 | Ocimum basilicum |
| MYRS1_ARATH | Q9ZUH4 | Arabidopsis thaliana |
| MYRS2_ARATH | Q9LRZ6 | Arabidopsis thaliana |
| MYRS_QUEIL | Q93X23 | Quercus ilex |
| TPSD2_ABIGR | Q24474 | Abies grandis |
| Q58GE8_IPSPI | Q58GE8 | Ips pini |
| (E)-β-Ocimene Synthase | | |
| (E.C. No. 4.2.3.B40) | | |
| Q5UB07_MEDTR | Q5UB07 | Medicago truncatula |
| Q672F7_LOTJA | Q672F7 | Lotus japonicas |
| Q84NC8_ANTMA | Q84NC8 | Antirrhinum majus |
| β-Farnesene Synthase | | |
| (E.C. No. 4.2.3.47) | | |
| FARS_ZEAMM | C7E5V7 | Zea mays subsp. Mexicana |
| FARS_MAIZE | Q2NM15 | Zea mays |
| TPSBF_MENPI | O48935 | Mentha piperita |
| ACSS_MAIZE | Q84ZW8 | Zea mays |
| FARS_ARTAN | Q9FXY7 | Artemisia annua |
| FARS_ZEAMH | C7E5V8 | Zea mays subsp. |
| | | Huehuetenangensis |
| FARS_ZEADI | C7E5V9 | Zea diploperennis |
| FARS_ZEAPE | C7E5W0 | Zea perennis |
| FARS_CITJU | Q94JS8 | Citrus junos |
| D7PCH9_PINSY | D7PCH9 | Pinus sylvestris |
| Q0J7R9_ORYSJ | QOJ7R9 | Oryza sativa subsp. japonica |

Dimethylallyl diphosphate may be converted to isoprene by a three-step isoprene biosynthetic pathway. As used herein, enzyme names are defined as follows (FIG. 4. B.). A 3-methyl-2-buten-1-ol synthase or 3-methyl-2-buten-1-ol synthase is an enzyme that catalyzes the conversion of dimethylallyl diphosphate to 3-methyl-2-buten-1-ol, also referred to herein as prenol. A 2-methyl-3-buten-2-ol isomerase is an enzyme that catalyzes the isomerization of 3-methyl-2-buten-1-ol to 2-methyl-3-buten-2-ol. A 2-methyl-3-buten-2-ol dehydratase is an enzyme that catalyzes the conversion of 2-methyl-3-buten-2-ol to isoprene.

In a first step, dimethylallyl diphosphate is converted to 3-methyl-2-buten-1-ol by a 3-methyl-2-buten-1-ol synthase. In a second step, 3-methyl-2-buten-1-ol is converted to 2-methyl-3-buten-2-ol by a 2-methyl-3-buten-2-ol isomerase. In a third step, 2-methyl-3-buten-2-ol is converted to isoprene by a 2-methyl-3-buten-2-ol dehydratase. The second and third steps may be catalyzed by a single, bi-functional enzyme with both 2-methyl-3-buten-2-ol isomerase and 2-methyl-3-buten-2-ol dehydratase activities.

The conversion of dimethylallyl diphosphate to 3-methyl-2-buten-1-ol (prenol) may be catalyzed by a phosphatase. Examples of such phosphatases include enzymes encoded by the Bacillus subtilis genes yqkG (nudF) and yhfR (Withers, S. T., Gottlieb, S. S., Lieu, B., Newman, J. D. and J. D. Keasling. 2007. Identification of isopentenol biosynthetic genes from Bacillus subtilis by a screening method based on isoprenoid precursor toxicity. Appl. Env. Microbiol. 73: 6277-6283), although other known phosphatases and coding sequences with predicted phosphatase activity, for example, the ytjC gene of E. coli, may be used. Table 22, below, provides examples of phosphatases for use in the conversion of dimethylallyl diphosphate to prenol.

TABLE 22

| | GenBank Ac- | |
|---|---|---|
| Locus | cession No. | Organism |
| BAA12639 (YqkG) | BAA12639 | Bacillus subtilis |
| CAA74541 (YhfR) | CAA74541 | Bacillus subtilis subsp. |
| | | subtilis Strain 168 |
| GPMB_ECOLI (YtjC) | POA7A2 | Escherichia coli K-12 |

The conversion of dimethylallyl diphosphate to 3-methyl-2-buten-1-ol may be catalyzed by a terpene synthase, e.g., a geraniol synthase or farnesol synthase or mutants thereof, for example. Table 23, below, provides examples of terpene synthases for use in the conversion of dimethylallyl diphosphate to 3-methyl-2-buten-1-ol.

TABLE 23

| | GenBank Ac- | |
|---|---|---|
| Locus | cession No. | Organism |
| Geraniol Synthase | | |
| AAR11765 | AAR11765 | Ocimum basilicum |
| ABB30216 | ABB30216 | Perilla citriodora |
| ABB30217 | ABB30217 | Perilla citriodora |
| ABB30218 | ABB30218 | Perilla frutescens |
| CAE52821 | CAE52821 | Cinnamomum tenuipile |
| Farnesol Synthase | | |
| ACSS_MAIZE | Q84ZW8 | Zea mays |
| ABJ16554 | ABJ16554 | Oryza sativa |

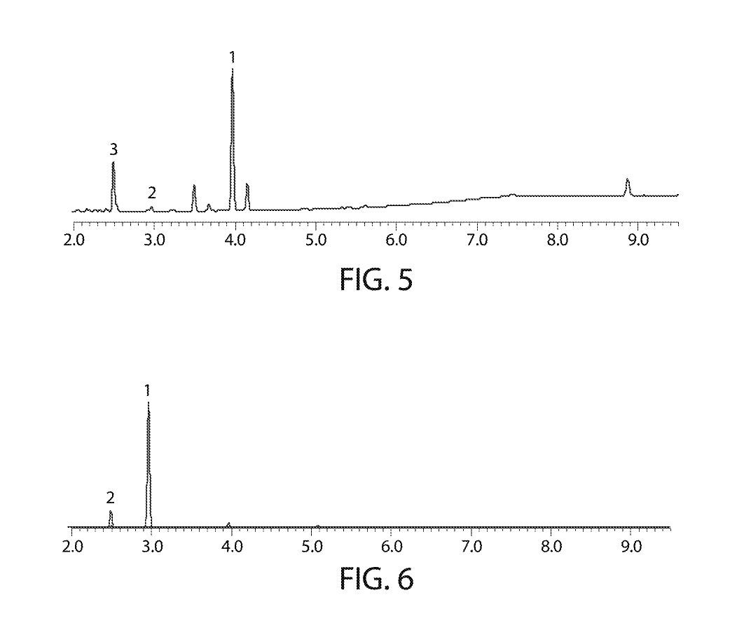

3-methyl-2-buten-1-ol is isomerized to 2-methyl-3-buten-2-ol by a 2-methyl-3-buten-2-ol isomerase. As used herein, a 2-methyl-3-buten-2-ol isomerase is an enzyme that converts 3-methyl-2-buten-1-ol (prenol) to 2-methyl-3-buten-2-ol in a reversible reaction. An example of such an enzyme is the linalool dehydratase-isomerase of Castellaniella defragrans strain 65Phen, GenBank accession number FR669447. This enzyme catalyzes the isomerization of 3-methyl-2-buten-1-ol to 2-methyl-3-buten-2-ol and the dehydration of 2-methyl-3-buten-2-ol to isoprene (Example 2, below, and FIG. 5). Orthologs, paralogs and nonorthologous gene displacements of linalool dehydratase-isomerase can be determined by methods well known to those skilled in the art.

As used herein, a 2-methyl-3-buten-2-ol dehydratase is an enzyme that converts 2-methyl-3-buten-2-ol to isoprene. An example of such an enzyme is the linalool dehydratase-isomerase of Castellaniella defragrans strain 65Phen, GenBank accession number FR669447. This enzyme is capable of catalyzing the dehydration of 2-methyl-3-buten-2-ol to isoprene (Example 3, below, and FIG. 6). Orthologs, paralogs and nonorthologous gene displacements of linalool dehydratase-isomerase can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity that is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

2-methyl-3-buten-2-ol dehydratase enzyme activity has also been identified in Aquincola tertiaricarbonis (Schuster, J., Schäfer, F., Hübler, N., Brandt, A., Rosell, M., Härtig, C., Harms, h., Müller, R. H. and T. Rohwerder. 2012. Bacterial degradation of tent-amyl alcohol proceeds via hemiterpene 2-methyl-3-buten-2-ol by employing the tertiary alcohol desaturase function of the Rieske nonheme mononuclear iron oxygenase MdpJ. J. Bact. 194: 972-981). The sequence of this 2-methyl-3-buten-2-ol dehydratase has not been reported.

Dimethylallyl diphosphate may be converted to isoprene by a two-step isoprene biosynthetic pathway (FIG. 4. C.). As used herein, a 2-methyl-3-buten-2-ol synthase is an enzyme that catalyzes the conversion of dimethylallyl diphosphate to 2-methyl-3-buten-2-ol.

As used herein, a 2-methyl-3-buten-2-ol synthase (also referred to as MBO synthase) is a nuclearly encoded, naturally occurring polypeptide found in some plant plastids, particularly in the chloroplast, that converts dimethylallyl diphosphate to 2-methyl-3-buten-2-ol, and derivatives (mutants) of polypeptides that naturally convert dimethylallyl diphosphate to 2-methyl-3-buten-2-ol. MBO synthases are characterized, in part, by an amino-terminal plastid targeting sequence that routes the polypeptide to the chloroplast. Upon translocation into the chloroplast, the transit peptide may be cleaved from the polypeptide to yield a mature protein that is smaller in molecular weight than the precursor protein. For overexpression of an exogenous MBO synthase in a microbial organism, it is preferable to express a truncated MBO synthase that approximates the mature form found in nature, rather than the precursor form. Essentially, the sequence encoding the transit peptide is removed from the MBO synthase coding sequence. While visual inspection may allow one skilled in the art to select where to truncate the isoprene synthase coding sequence, computer-based algorithms such as ChloroP 1.1 can be used to help predict which amino acids belong to the transit peptide (Emanuelsson, O., Nielsen, H., G. von Heijne. 1999. ChloroP, a neural network-based method for predicting chloroplast transit peptides and their cleavage sites. Protein Sci. 8: 978-984). An example of an MBO synthase is found in Pinus sabiniana, with the GenBank accession number AEB53064.1.

The conversion of dimethylallyl diphosphate to 2-methyl-3-buten-2-ol may be catalyzed by a terpene synthase, e.g., a linalool synthase (E.C. No. 4.2.3.25 or 4.2.3.26) or nerolidol synthase or mutants thereof, for example. Table 24, below, provides examples of terpene synthases for use in the conversion of dimethylallyl diphosphate to 2-methyl-3-buten-2-ol.

TABLE 24

| | GenBank Ac- | |
|---|---|---|
| Locus | cession No. | Organism |
| S-Linalool Synthase | | |
| LIS_CLABR | Q96376 | Clarkia breweri |
| LINS_ARATH | Q84UV0 | Arabidopsis thaliana |
| C0KWV3_9LAMI | C0KWV3 | Perilla setoyensis |
| C0KWV5_PERFR | C0KWV5 | Perilla frutescens |
| | | var. hirtella |
| C0KWV7_PERFR | C0KWV7 | Perilla frutescens |
| | | var. hirtella |
| D4N3A0_9ERIC | D4N3A0 | Actinidia arguta |
| D4N3A1_9ERIC | D4N3A1 | Actinidia polygama |
| R-Linalool Synthase | | |
| LLOS1_ARTAN | Q9SPN0 | Artemesia annua |
| LLOS_OCIBA | Q5SBP3 | Ocimum basilicum |
| LLOS5_ARTAN | Q9SPN1 | Artemesia annua |
| LLOS_MENAQ | Q8H2B4 | Mentha aquatica |
| Q1XBU5_SOLLC | Q1XBU5 | Solatium lycopersicum |
| (3S, 6E)-Nerolidol Synthase | | |
| Q5UB06_MEDTR | Q5UB06 | Medicago trunculata |

The conversion of 2-methyl-3-buten-2-ol to isoprene may be catalyzed by a 2-methyl-3-buten-2-ol dehydratase as described above. The 2-methyl-3-buten-2-ol dehydratase may be a bi-functional enzyme with both 2-methyl-3-buten-2-ol isomerase and 2-methyl-3-buten-2-ol dehydratase activities, such as the linalool dehydratase-isomerase described above, or the enzyme may encode only the 2-methyl-3-buten-2-ol dehydratase activity without a 2-methyl-3-buten-2-ol isomerase activity.

The conversion of glycerol to undesirable co-products may be reduced or eliminated by mutations of one or more genes. In general, expression of these genes may result in the routing of carbon from glycerol to undesirable co-products, or create an unfavorable reduction/oxidation state within the host cell, reducing the yield of isoprene from glycerol. Examples of desirable genes/enzyme activities to be reduced or eliminated include: lactate dehydrogenase (for example, the ldhA or lldD genes of E. coli), succinate dehydrogenase (for example, the frdABCD genes of E. coli), acetate kinase (the ackA gene of E. coli), phosphate acetyltransferase (the pta gene of E. coli), pyruvate oxidase (the poxB gene of E. coli), or alcohol dehydrogenase (the adhE gene of E. coli).

### Strains, Plasmids and Gene Abbreviations

In the following examples of embodiments of the current invention, the common E. coli cloning strains DH1OB and DH5a (GC5) were used during construction of all vectors. For testing the plasmid constructs, either BL21(DE3), MG1655, or the MG1655 derivative strain LA02 was used as the host strain. DNA acquired through complete synthesis was received already transformed in DH10B. Vectors that were constructed in house were transformed into chemically competent DH5a cells (GC5, Gene Choice, available from Sigma-Aldrich Co. LLC). Wild type MG1655 was obtained from the University of Wisconsin E. coli Genome Project (https://www.genome.wisc.edu). MG1655 was made electrocompetent and electroporated following the protocol from the MicroPulser Electroporation Apparatus Operating Instructions and Applications Guide (Bio-Rad catalog number 165-2100), except that LB without salt was used to grow up the culture in making cells electrocompetent. Strain genotypes are found in Table 25.

TABLE 25
STRAINS

| Strain | Genotype | Reference |
|---|---|---|
| MG1655 | Wild type E. coli (F⁻ λ⁻ ilvR⁻ rfb-50 rph-1) | ATCC^{‡} 47076 |
| LA02 | MG1655 ΔackA Δpta ΔfrdA ΔadhE | This work. |
| BL21(DE3) | fhuA2 [lon] ompT gal (λ DE3) [dcm] ΔhsdS | New England Biolabs, Inc. |
| | λ DE3 = λ sBamHIo ΔEcoRI-B int::(lacI::PlacUV5::T7 | (www.neb.com) |
| | gene1) i21 Δnin5 | |
| DH10B | E. coli (F⁻ endA1 recA1 galE15 galK16 nupG rpsL ΔlacX74 | DNA2.0, www.dna20.com |
| | Φ80lacZΔM15 araD139 Δ(ara, leu)7697 mcrA Δ(mrr- | |
| | hsdRMS-mcrBC) λ⁻⁾ | |
| GC5 (DH5α) | E. coli (F⁻ φ 801acZ Δ M15 Δ(lacZYA-argF)U169 recA1 | Sigma-Aldrich Co. LLC, |
| | endA1 hsdR17(rk⁻, mk⁺) phoA supE44 thi-1 gyrA96 relA1 | www.sigmaaldrich.com |
| | λ⁻ tonA) | |
| TRANSFORMAX ™ | F⁻ mcrA Δ(mrr-hsdRMS-mcrBC) φ80dlacZΔM15 ΔlacX74 | Epicentre Biotechnologies |
| EC100D ™ pir⁺ | recA1 endA1 araD139 Δ(ara, leu)7697 galU galK λ⁻ rpsL | (Illumina, Inc.; www.epibio.com) |
| | (Str^{R}) nupG pir⁺ (DHFR) | |

^{‡}American Type Culture Collection

TABLE 26
PLASMIDS

| Plasmid | Features | Source |
|---|---|---|
| pZS.glpK.glpD | E. coli glpK and glpD genes under the control of P_{LtetO-1} (tetR, | Mazumdar, S., Clomburg, J. M., |
| | oriR SC101*) | and R. Gonzalez. 2010. |
| | | Escherichia coli strains |
| | | engineered for homofermentative |
| | | production of D-lactic acid from |
| | | glycerol. Appl. Env. Microbiol. |
| | | 76: 4327-4336. |
| pZS.adhEp.glpK.glpD | E. coli glpK and glpD genes under the control of adhE | This work |
| | promoter (tetR, oriR SC101*) | |
| pR6Kan | Kanamycin-resistant derivative of pGP704 | Orchard, S. S., and H. Goodrich- |
| | | Blair. 2005. Pyrimidine |
| | | nucleoside salvage confers an |
| | | advantage to Xenorhabdus |
| | | nematophila in its host |
| | | interactions. Appl. Environ. |
| | | Microbiol. 71: 6254-6259 |
| pR6KT-ΔldhA | pR6Kan derivative for deleting ldhA in E. coli | Glycos Biotechnologies, Inc. |
| pR6KT-ΔfrdA | pR6Kan derivative for deleting frdA in E. coli | Glycos Biotechnologies, Inc. |
| pR6KT-ΔackAΔpta | pR6Kan derivative for deleting ackA and pta in E. coli | Glycos Biotechnologies, Inc. |
| pR6KT-ΔadhE1Kb | pR6Kan derivative for deleting adhE in E. coli | Glycos Biotechnologies, Inc. |
| pJexpress401 | Bacterial expression vector, Rep_{pUC} Km^{R} | DNA2.0 (www.dna20.com) |
| pJexpress404 | Bacterial expression vector, Rep_{pUC} Ap^{R} | DNA2.0 (www.dna20.com) |
| pJ404-LDI | | Glycos Biotechnologies, Inc. |
| pJ404-SAAT | | Glycos Biotechnologies, Inc. |
| pJ401-MBO1.IDI | | Glycos Biotechnologies, Inc. |
| pJ401-MBO1.IDI.LDI | | Glycos Biotechnologies, Inc. |
| pGA31R-MCS | Rep_{p15A} Cm^{R} tetR MCS | DNA2.0 (www.dna20.com) |
| pGE21R-MCS | Rep_{ColE1} Km^{R} tetR P_{LtetO-1} MCS T1 | |
| pJ248-mvaES | Rep_{pUC} Ap^{R} Gent^{R} mvaES_{Ef, optEc} | DNA2.0 (www.dna20.com) |
| pJ241-MK.PMK.MPD.IDI | Rep_{pUC} Km^{R} T1 P_{LtetO-1} MK_{Mj, optEc} PMK_{Ef, optEc} MPD_{Sc, optEc} | DNA2.0 (www.dna20.com) |
| | IDI_{Ec, optEc} T1 | |
| pUC57-ispS | Rep_{ColE1} Ap^{R} P_{lac} ispS_{Pa, optEc} | GenScript, Inc. |
| pGB1004 | Rep_{ColE1} Km^{R} tetR P_{LtetO-1} ispS_{Pa, optEc} T1 | Glycos Biotechnologies, Inc. |
| pGB1008 | Rep_{p15A} Cm^{R} tetR P_{LtetO-1} mvaES_{Ef, optEc} T1 | Glycos Biotechnologies, Inc. |
| pGB1012 | Rep_{ColE1} Km^{R} tetR P_{LtetO-1} ispS_{Pa, optEc} IDI_{Ec, optEc} T1 | Glycos Biotechnologies, Inc. |
| pGB1030 | Rep_{p15A} Cm^{R} tetR P_{LtetO-1} mvaES_{Ef, optEc} T1 P_{LtetO-1} MK_{Mj, optEc} | Glycos Biotechnologies, Inc. |
| | PMK_{Ef, optEc} MPD_{Sc, optEc} IDI_{Ec, optEc} T1 | |
| pGB1030-GlyOH | pGB1030 P_{adhEp} | Glycos Biotechnologies, Inc. |

Table 26 Notes:
Ef = Enterococcus faecalis ATCC 700802; Mj = Methanocaldococcus jannaschii; Sc = Saccharomyces cerevisiae S288C; Ec = Escherichia coli MG1655; Pa = Populus alba; optEC = codon-optimized for expression in E. coli; MCS = multiple cloning site; Rep = origin of replication (ColE1, p15A or pUC); P_{LtetO-1} = anhydrotetracycline-inducible promoter; P_{adhEp} = constitutive promoter fragment from E. coli adhE gene; T1 = terminator T1 of the rrnB operon of E. coli.

### Example 1

### Improving Glycerol Dissimilation

This working example demonstrates the improvement of glycerol dissimilation by overexpression of glpK and glpD in engineered E. coli. In this example, the rate of production of lactic acid is used as a proxy for flux of glycerol through the key metabolic intermediate dihydroxyacetone phosphate.

The E. coli strain used was LA02 (MG1655 AackA Apta AfrdA AadhE) containing plasmid pZS.adhEp.glpK.glpD. The strain was constructed as follows:

To create the basic suicide vector used to delete genes from the E. coli chromosome, the R6Kγ origin of replication, kanamycin marker and multiple cloning site of plasmid pR6Kan (Orchard, S. S., and H. Goodrich-Blair. 2005. Pyrimidine nucleoside salvage confers an advantage to Xenorhabdus nematophila in its host interactions. Appl. Environ. Microbiol. 71:6254-6259) were amplified by polymerase chain reaction (PCR) using the following primers:

Primer 1:

[SEQ ID NO: 1]

5′-ATCGAGCTCAACCATCATCGATGAATTGC-3′
Primer 2:

[SEQ ID NO: 2]

5′-TCTGGTACCCTGAAGCTTGCATGCC-3′

The first primer introduces a Sad restriction enzyme site while the second primer introduces a KpnI restriction enzyme site.

An approximately 500 base pair (bp) fragment upstream of the E. coli ldhA gene was PCR amplified using the following primers:

Primer 1:

[SEQ ID NO: 3]

5′-ATAGAGCTCCCACCAGATAACGG-3′
Primer 2:

[SEQ ID NO: 4]

5′-GCGCTCGAGTTTCATAAGACTTTCTCC-3′

The first primer introduces a Sad restriction enzyme site while the second primer introduces a XhoI restriction site.

An approximately 500 base pair fragment downstream of the E. coli ldhA gene was PCR amplified using the following primers:

Primer 1:

[SEQ ID NO: 5]

5′-GAC CTC GAG CTG GTT TAA TCT TGC CG-3′
Primer 2:

[SEQ ID NO: 6]

5′-CGT GGT ACC TTC TTT CAG CAT TTC G-3′

The first primer introduces a XhoI restriction enzyme site while the second primer introduces a Sad restriction site.

The three PCR products were restriction-digested with Sad, XhoI and/or KpnI restriction enzymes as appropriate, and the resulting fragments were used in a trimolecular ligation reaction with the QUICK LIGASE KIT™ (New England Biolabs, Ipswich, Mass.). Restriction enzymes and buffer components were removed from the ligation reaction using the DNA CLEAN AND CONCENTRATOR KIT™ (Zymo Research, Irvine, Calif.) before electroporation into TransforMax EC100D pir+ electrocompetent cells (Epicentre Biotechnologies, Madison, Wis.). Transformants were selected on LB plates containing 50 μg/ml kanamycin and the correct plasmid identified by restriction digestion. The resulting intermediate plasmid was then further modified by introduction of the tetRA locus as a counterselectable marker as follows: the intermediate plasmid was restriction digested using BamHI, while the tetRA locus was PCR amplified using primers P7 and P8; the two DNA fragments were joined using the IN-FUSION CLONING SYSTEM™ (Clontech, Mountain View, Calif.), diluted five-fold with water, then electroporated into TransforMax EC100D pir+electrocompetent cells. Transformants were selected on LB plates containing 50 μg/mlkanamycin and the correct plasmid identified by restriction digestion. This plasmid is named pR6KT-ΔldhA.

Plasmid pR6KT-ΔfrdA was constructed as follows. An approximately 448 base pair fragment upstream of the E. coli frdA gene was PCR amplified using the following primers:

Primer 1:

[SEQ ID NO: 7]

5′-ATTGAGCTCGGTTACCGTCGCC-3′
Primer 2:

[SEQ ID NO: 8]

5′-GCCATTCGCCTTCTCGAGCACGACATTCCTCC-3′

The first primer introduces a Sad restriction enzyme site while the second primer introduces a XhoI restriction site.

An approximately 471 base pair fragment downstream of the E. coli frdA gene was PCR amplified using the following primers:

Primer 1:

[SEQ ID NO: 9]

5′-GGAGGAATCTCGTGCTCGAGAAGGCGAATGGC-3′
Primer 2:

[SEQ ID NO: 10]

5′-AGCGGTACCACAGTTGATGCAACC-3′

The first primer introduces a XhoI restriction enzyme site while the second primer introduces a KpnI restriction site. The two resulting PCR products were joined using overlap extension PCR (OE-PCR) with the following primers:

Primer 1:

[SEQ ID NO: 11]

5′-ATTGAGCTCGGTTACCGTCGCC-3′
Primer 2:

[SEQ ID NO: 12

5′-AGCGGTACCACAGTTGATGCAACC-3′

The resulting PCR product was digested with Sad and KpnI. Plasmid pR6KT-ΔldhA was restriction digested with Sad and KpnI followed by purification by agarose gel electrophoresis. The band corresponding to the vector backbone was recovered from the agarose using the GEL DNA RECOVERY KIT™ (Zymo Research). The two restriction-digested DNA fragments corresponding to the vector backbone, and the fused upstream and downstream regions of frdA, were used in a ligation reaction with the QUICK LIGASE KIT™ (New England Biolabs). Restriction enzymes and buffer components were removed from the ligation reaction using the DNA CLEAN AND CONCENTRATOR KIT™ (Zymo Research) before electroporation into TransforMax EC100D pir+ electrocompetent cells (Epicentre Biotechnologies). Transformants were selected on LB plates containing 50 μg/mlkanamycin and the correct plasmid identified by restriction digestion.

pR6KT-ΔackAΔpta. Plasmid pR6KT-ΔackAΔpta was constructed as follows. An approximately 462 base pair fragment upstream of the E. coli ackA gene was PCR amplified using the following primers:

Primer 1:

[SEQ ID NO: 13]

5′-ACGGAGCTCATCTTGATAACGCGAT-3′
Primer 2:

[SEQ ID NO: 14]

5′-TACCTCGAGCATGGAAGTACCTATAATTG-3′

The first primer introduces a Sad restriction enzyme site while the second primer introduces a XhoI restriction enzyme site. The resulting PCR product was restriction digested with Sad and XhoI.

An approximately 473 base pair fragment downstream of the E. coli pta gene was PCR amplified using the following primers:

Primer 1:

[SEQ ID NO: 15]

5′-GGATAAACCGTGTCCCTCGAGCAGCAGTAATCTCG-3′
Primer 2:

[SEQ ID NO: 16]

5′-ACTGGTACCTCTAGAAGTTCATCAGGCC-3′

The first primer introduces a XhoI restriction enzyme site while the second primer introduces a KpnI restriction enzyme site. The resulting PCR product was restriction digested with XhoI and KpnI.

Plasmid pR6KT-ΔldhA was restriction digested with Sad and KpnI followed by purification by agarose gel electrophoresis. The band corresponding to the vector backbone was recovered from the agarose using the GEL DNA RECOVERY KIT™ (Zymo Research). The three restriction-digested DNA fragments corresponding to the vector backbone, the upstream region of ackA, and the downstream region of pta were used in a trimolecular ligation reaction with the QUICK LIGASE KIT™ (New England Biolabs, Ipswich, Mass.). Restriction enzymes and buffer components were removed from the ligation reaction using the DNA CLEAN AND CONCENTRATOR KIT™ (Zymo Research) before electroporation into TransforMax EC100D pir+ electrocompetent cells (Epicentre Biotechnologies). Transformants were selected on LB plates containing 50 μg/ml kanamycin and the correct plasmid identified by restriction digestion.

Plasmid pR6KT-ΔadhE1 kb was constructed as follows. An approximately 1000 base pair fragment upstream of the E. coli adhE gene was PCR amplified using the following primers:

Primer 1:

[SEQ ID NO: 17]

5′-ATAGAGCTCAGCGAACAATGCAATAGCC-3′
Primer 2:

[SEQ ID NO: 18]

5′-CAGCGCTACTGATTAAGCTGTACAAGTAACAGCCATA
ATGCTCTCC-3′

The first primer introduces a Sad restriction enzyme site while the second primer introduces a BsrGI restriction site.

An approximately 1000 base pair fragment downstream of the E. coli adhE gene was PCR amplified using the following primers:

Primer 1:

[SEQ ID NO: 19]

5′-GGAGAGCATTATGGCTGTTACTTGTACAGCTTAATCAGT
AGCGCTG-3′
Primer 2:

[SEQ ID NO: 20]

5′-ACAGCATGCGAACACCACTTCCAGAGC-3′

The first primer introduces a BsrGI restriction enzyme site while the second primer introduces a SphI restriction site. The two resulting PCR products were joined using overlap extension PCR (OE-PCR) with the following primers:

Primer 1:

[SEQ ID NO: 21]

5′-ATAGAGCTCAGCGAACAATGCAATAGCC-3′
Primer 2:

[SEQ ID NO: 22]

5′-ACAGCATGCGAACACCACTTCCAGAGC-3′

The resulting PCR product and plasmid pR6KT-ΔldhA were restriction digested with Sad and SphI followed by purification by agarose gel electrophoresis. The bands corresponding to the PCR fragment and vector backbone were recovered from the agarose using the GEL DNA RECOVERY KIT™ (Zymo Research). The two restriction-digested DNA fragments corresponding to the vector backbone, and the fused upstream and downstream regions of adhE, were used in a ligation reaction with the QUICK LIGASE KIT™ (New England Biolabs). Restriction enzymes and buffer components were removed from the ligation reaction using the DNA CLEAN AND CONCENTRATOR KIT™ (Zymo Research) before electroporation into TransforMax EC100D pir+ electrocompetent cells (Epicentre Biotechnologies). Transformants were selected on LB plates containing 50 μg/ml kanamycin and the correct plasmid identified by restriction digestion.

Gene deletions in E. coli were made using the approach of Metcalf and colleagues (Metcalf, W. W., Jiang, W., Daniels, L. L., Kim, S. K., Haldiman, A. and B. L. Wanner. 1996. Conditionally replicative and conjugative plasmids carrying lacZa for cloning, mutagenesis, and allele replacement in bacteria. Plasmid 35: 1-13). E. coli MG1655 strains with various genes deleted were made using suicide vectors. pR6KT-based gene knock-out plasmids were purified from TransforMax EC100D using standard plasmid DNA purification techniques and concentrated to approximately 1 μg/ml or greater. Concentrated plasmid DNA was electroporated into the pir⁻ recipient strain. Transformants were selected on LB agar plates containing 15 μg/ml tetracycline and 2.5 mM Na₄P₂O₇. After confirming integration by testing for resistance to tetracycline, integration into the correct locus was confirmed by PCR. Transformants with the knock-out plasmid integrated into the proper locus were restreaked onto LB agar plates without antibiotic selection to provide the opportunity for chromosomal rearrangement to resolve the gene duplication. Individual colonies from the LB agar plate were then restreaked onto tetracycline-sensitive-selection agar (TSS) and incubated for two or more days at 30° C. Large colonies from the TSS agar plates were then tested for sensitivity to tetracycline and kanamycin using LB agar plates containing either tetracycline (15 μg/ml) or kanamycin (50 μg/ml). As resolution of the gene duplication can either generate a gene deletion or recreate the wild-type locus, colonies sensitive to both kanamycin and tetracycline were then tested for the desired gene deletion using PCR. Further confirmation was provided by restriction digest of the PCR product with either XhoI or BsrGI (as appropriate).

TSS agar plates were made as follows: 4.347 g NaH₂PO₄ was mixed with 100 mL distilled water. To this solution, the following chemicals were added: 3 ml of fusaric acid, 2 mg/ml; 2.5 mL ZnCl₂, 20 mM; and 0.5 ml anhydrotetracycline, 5 mg/mL. This buffer solution was sterilized by nanofiltration. 2.5 g tryptone, 2.5 g yeast extract, 5 g sodium chloride, and 7.5 g agar were mixed with 400 mL distilled water and autoclaved. Once the agar solution cooled to approximately 45° C., it was mixed with 100 mL of the buffer solution. This final buffer/agar mixture was then poured into 100 mm-diameter petri plates.

Using the above gene deletion method, the ackA/pta, frdA and adhE genes were sequentially deleted from MG1655 to create LA02.

Plasmid pZS.adhEp.glpK.glpD was constructed by inserting an adhE promoter at the XhoI and KpnI restriction sites of plasmid pZS.glpK.glpD, replacing the PLteto-1 promoter. The adhE promoter was used because it is a constitutive promoter. The adhE promoter was amplified by PCR using genomic DNA of E. coli MG1655 as the template and the following primers:

Primer 1:

[SEQ ID NO: 23]

5′-CGCCTCGAGGGTTAGCTCCGAAGCAAAAGC-3′
Primer 2:

[SEQ ID NO: 24]

5′-CGCGGTACCAATGCTCTCCTGATAATGTTAAAC-3′.

The resulting PCR product was digested with XhoI and KpnI. Likewise, pZS.glpK.glpD was digested with XhoI and KpnI. The two restriction digested fragments corresponding to the vector backbone and the adhE promoter was purified by agarose gel electrophoresis and fused together in a ligation reaction using the QUICK LIGASE KIT™ (NEB). The ligation reaction was purified using the DNA CLEAN AND CONCENTRATOR KIT™ (Zymo Research) to reduce PEG concentrations before electroporation into GC5 electrocompetent cells. Transformants were selected on LB plates containing 20 μg/ml chloramphenical and the correct plasmid identified by restriction digestion.

Strain LA02 was transformed with plasmid pZS.adhEp.glpK.glpD using electroporation. A single colony was cultured for 16 hours at 37° C. in 5 mL of LB medium supplemented with 20 μg/mL chloramphenicol. 0.5 mL of the culture was added to 0.5 -mL of sterile glycerol, mixed well, and stored at −80° C.

All experiments were routinely started from strains stored at −80° C. as glycerol stocks. LA02 not bearing plasmids was streaked from the appropriate glycerol stock onto LB-agar plates (LB medium is 10 g/L NaCl, 5 g/L yeast extract, 10 g/L tryptone, supplemented with 15 g/L agar). LA02 (pZS.adhEp.glpK.glpD) was streaked from the appropriate glycerol stock onto LB-agar plates supplemented with 20 μg/mL chloramphenicol. Individual colonies of LA02 (pZS.adhEp.glpK.glpD) were used to inoculate 5 mL of Luria Bertani broth supplemented with 20 μg/mL chloramphenicol, 20 g/L glycerol and 5 g/L CaCO₃ in a 25-mL Erlenmeyer flask. Flasks were incubated for 16 hours at 37° C. in a Lab Companion SI-600R incubating shaker set at 175 rpm.

The 16-hour cultures were used to seed 400-mL cultures of Luria Bertani broth supplemented with 50 g/L CaCO₃, 20 μg/mL chloramphenicol, and 60 g/L glycerol in a 0.5-L working volume fermentor (Ward's Natural Science, Rochester. N.Y.) with independent control of temperature, pH, and stirrer speed. All experimental cultures were seeded with sufficient cells to achieve an initial optical density at 600 nm of 0.1. Temperature was maintained at 37° C. pH was maintained at 7.0 using 5 N NaOH. The stirrer speed was maintained at 200 rpm. The cultures were aerated at 10 mL/min using air, sufficient to achieve a k_{L}a of 100 h⁻¹. 5-mL samples were withdrawn from the cultures at 0-, 24-, 48- and 72-hour time points. The samples were used to measure cell growth, glycerol consumption, and metabolite production. Cell growth was determined by measuring the optical absorbance of a 1 to 10 dilution of the culture at 600 nm in a Biochrom Libra S22 spectrophotometer.

Glycerol consumption and metabolite production were quantified by HPLC analysis. All samples were filtered through a 0.22 μm polyvinylidene fluoride syringe filter (Millipore) prior to HPLC analysis. Routinely, 10-μL of filtered fermentation medium was injected onto an HPLC (LC-10AD vp, Shimadzu, Kyoto, Japan] fitted with a Rezex ROA-Organic Acid H⁺ (8%) 150×7.8 mm column (Phenomenex, Torrance, Calif.) at 65° C. with a mobile phase of 2.5 mM H₂SO₄ operated under isocratic conditions at a flow rate of 0.6 mL/minute. Metabolites were detected via a refractive index detector (RID-10A, Shimadzu).

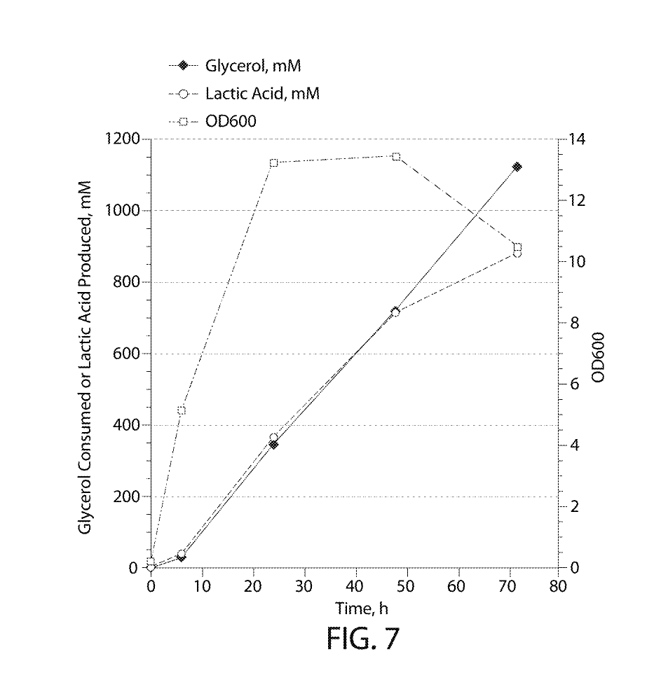

Typical results are presented in FIG. 7. Over the 72-hour period, LA02 (pZS.adhEp.glpK.glpD) consumed glycerol at the average rate of 1.2 mM/L/h/OD and lactic acid was produced at the average rate of 0.94 mM/L/h/OD. The final titer of lactic acid reached 79.32 g/L, while approximately 103 g/L glycerol was consumed.

### Example 2

### Microorganism for the Production of Isoprene from 3-Methyl-2-Buten-1-OL

This working example shows the production of isoprene from 3-methyl-2-buten-1-ol by a non-naturally occurring microorganism expressing one or more exogenous genes of an isoprene biosynthetic pathway.

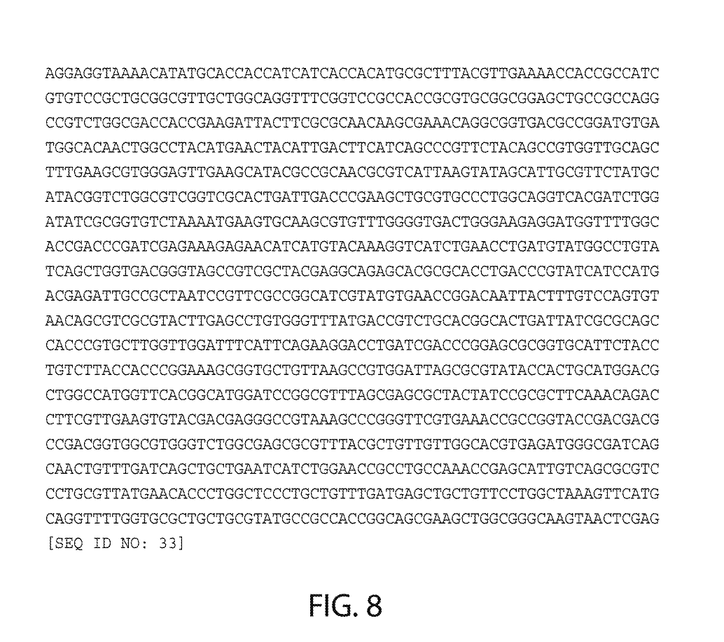

The plasmid pJ404-LDI was constructed by DNA2.0 (Menlo Park, Calif.) using the codon-optimized sequence of the linalool dehydratase-isomerase (LDI) of Castellaniella defragrans strain 65Phen (FIG. 8). The LDI coding sequence was codon-optimized for expression in E. coli using the proprietary algorithms of DNA2.0, synthesized and inserted into the plasmid expression vector pJexpress404. The resulting plasmid, pJ404-LDI, was electroporated into E. coli BL21(DE3) electrocompetent cells.

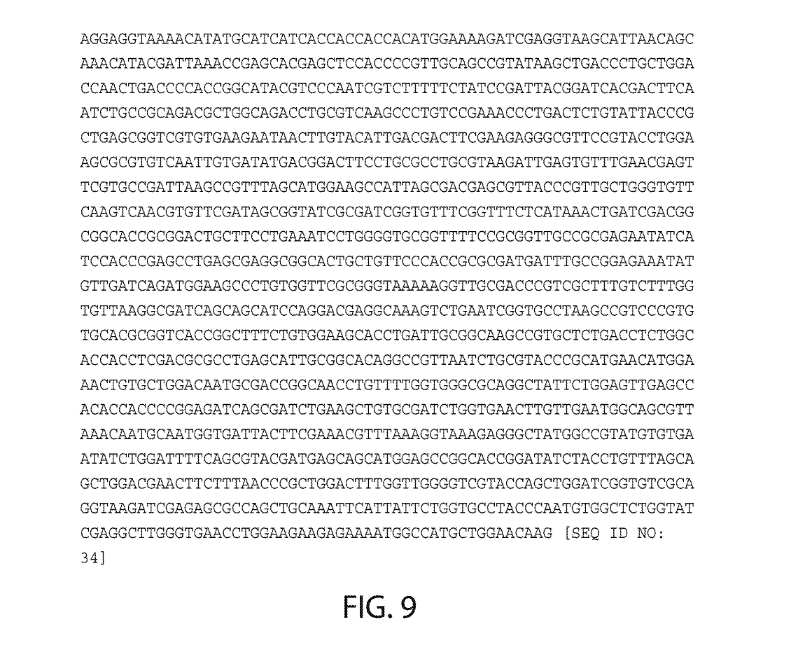

Plasmid pJ404-SAAT was constructed by DNA2.0 (Menlo Park, Calif.) using the codon-optimized sequence of the strawberry acyl-CoA transferase (SAAT) (FIG. 9). The SAAT coding sequence was codon-optimized for expression in E. coli using the proprietary algorithms of DNA2.0, synthesized and inserted into the plasmid expression vector pJexpress404. The resulting plasmid, pJ404-SAAT, was electroporated into E. coli BL21(DE3) electrocompetent cells. pJ404-SAAT was used as a negative control.

Transformants of BL21(DE3) harboring either pJ404-LDI or pJ404-SAAT were selected on Luria-Bertani (LB)-agar plates (10 g/L yeast extract, 5 g/L Bacto Tryptone, 10 g/L sodium chloride, 15 g/L Bacto Agar) containing 100 μg/ml ampicillin. The flask was incubated for 16 hours at 37° C. in a rotary shaking incubator. After 16 hours, the culture was diluted using LB broth containing 100 μg/ml ampicillin to an optical density of 0.16 at 600 nm.

A single colony of BL21(DE3) harboring pJ404-LDI or pJ404-SAAT from the LB-agar plates was used to inoculate 10 milliliter aliquots of LB broth (10 g/L yeast extract, 5 g/L Bacto Tryptone, 10 g/L sodium chloride) containing 100 μg/ml ampicillin contained in 125-mL Erlenmeyer flasks. Flasks were incubated for 16 hours at 37° C. in a rotary shaking incubator. After 16 hours, the resulting cultures were diluted using fresh LB broth containing 100 μg/ml ampicillin to an optical density of 0.16 at 600 nm. 50 milliliters of the diluted cultures were placed in 300-mL Erlenmeyer flasks and incubated at 37° C. in a rotary shaking incubator until the optical density at 600 nm reached approximately 0.6, typically 90 minutes. Four milliliters of the resulting cultures were then placed into 20-mL gas chromatography headspace vials. 3-methyl-2-buten-1-ol was added to a final concentration of 1 mM, IPTG (Isopropyl(3-D-1-thiogalactopyranoside) was added to 1 mM, and the cultures were grown for an additional 16 hours at 37° C. with shaking.

Isoprene was measured using headspace analysis on an Agilent 7890A GC equipped with a CTC-PAL autosampler and a FID. Headspace vials (20 mL) were incubated at 50° C. with agitation at 500 rpm for 2 minutes. Then 1 mL of the headspace was removed using a heated headspace syringe at 50° C. and injected into the GC inlet (250° C., split of 20:1). Samples were analyzed using a FID detector set at 300° C., with a helium carrier gas flow rate of 2 ml/min through a DB-624 30 m×530 μm×3 μm column (J&W Scientific), and an oven program of 85° C. for 5.25 minutes. The isoprene concentration in samples was calculated from calibration curves generated from isoprene calibration gas standards analyzed under the same GC/FID method. The isoprene product was also confirmed by headspace GC/MS using an Agilent 7890A GC equipped with a 5975C MSD and a CTC-PAL autosampler. Headspace vials were incubated at 85° C. with agitation at 600 rpm for 5 minutes. Then 1 mL of the headspace was removed using a heated headspace syringe at 85° C. and injected into the GC inlet (250° C., split of 25:1). The GC/MS method used helium as the carrier gas at 1 mL/min through a HP-5MS 30 m×250 μm×0.25 μm column (J&W Scientific), an oven program of 35° C. for 4 minutes, then ramped 25° C./min to 150° C., a MS source temperature of 230° C., and a quadrupole temperature of 150° C. The mass spectrometer was operated in scan mode from 25 to 160 mass units. The isoprene peak was identified by the NIST 11 MS Library, as well as comparison against an authentic sample (135 ppm isoprene, 135 ppm carbon dioxide in dry nitrogen gas, Matheson TRIGAS, Houston, Tex.).

3-methyl-2-buten-1-ol and 2-methyl-3-buten-1-ol were measured using headspace analysis on an Agilent 7890A GC equipped with a CTC-PAL autosampler and a FID. Headspace vials (20 mL) were incubated at 85° C. with agitation at 600 rpm for 5 minutes. Then 1 mL of the headspace was removed using a heated headspace syringe at 85° C. and injected into the GC inlet (250° C., split of 25:1). Samples were analyzed using a FID detector set at 350° C., with a helium carrier gas flow rate of 3 ml/min through at DB-624 30 m×530 μm×3 μm column (J&W Scientific), and an oven program of 90° C., then ramping 20° C./min to 230° C. for 3 minutes. The 3-methyl-2-buten-1-ol and 2-methyl-3-buten-2-ol concentrations in samples were calculated from calibration curves generated from diluted standards of each compound analyzed under the same GC/FID method.

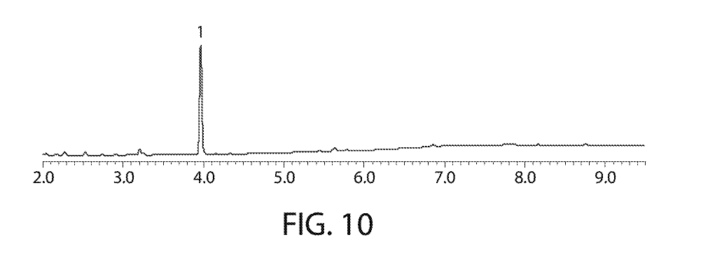

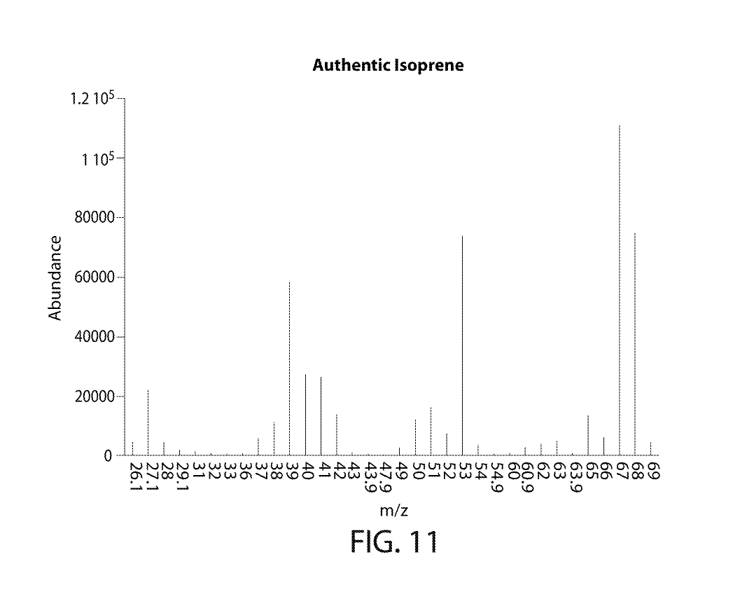

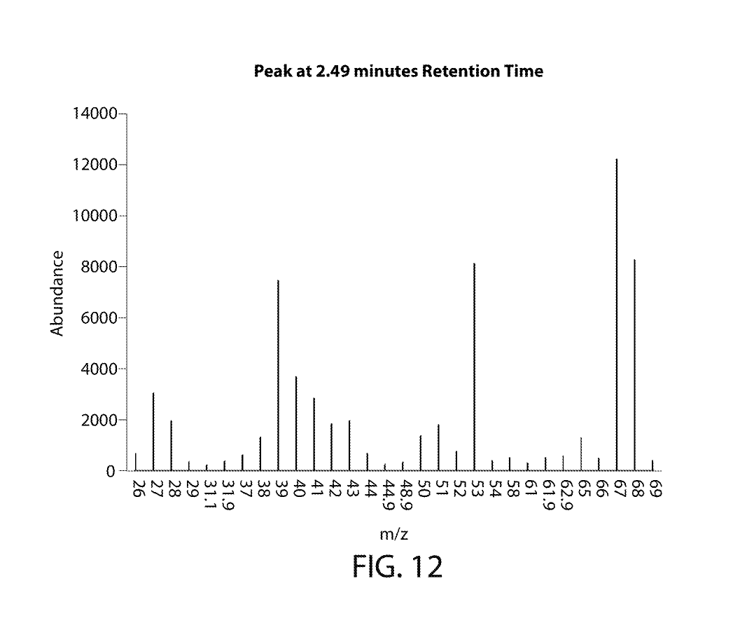

The results of this example are presented in FIG. 5 and FIG. 10. LB broth containing 1 mM 3-methyl-2-buten-1-ol with E. coli cells omitted showed a peak at 3.96 minutes corresponding to 3-methyl-2-buten-1-ol (FIG. 10). Similarly, cultures containing 1 mM 3-methyl-2-buten-1-ol and BL21(DE3) cells harboring pJ404-SAAT showed a peak at 3.96 minutes corresponding to 3-methyl-2-buten-1-ol, and an additional peak corresponding to the aldehyde 3-methyl-2-buten-al (prenal, data not shown). In contrast, cultures containing 1 mM 3-methyl-2-buten-1-ol and BL21(DE3) cells harboring pJ404-LDI converted 3-methyl-2-buten-1-ol to 2-methyl-3-buten-2-ol and isoprene, corresponding to peaks at 2.96 minutes and 2.49 minutes, respectively (FIG. 5). This demonstrates that E. coli cells harboring pJ404-LDI isomerize 3-methyl-2-buten-1-ol to 2-methyl-3-buten-2-ol and dehydrate 2-methyl-3-buten-2-ol to isoprene. FIG. 11 presents the GC/MS analysis of an authentic isoprene sample; FIG. 12 presents the GC/MS analysis of the peak with a 2.49 minute retention time, with the same fragmentation pattern as authentic isoprene shown in FIG. 5.

### Example 3

### Microorganism for the Production of Isoprene from 2-Methyl-3-Buten-2-Ol

This working example shows the production of isoprene from 2-methyl-3-buten-2-ol by a non-naturally occurring microorganism expressing one or more exogenous genes of an isoprene biosynthetic pathway.

A single colony of BL21(DE3) harboring pJ404-LDI or pJ404-SAAT from LB-agar plates was used to inoculate 10 milliliter aliquots of LB broth (10 g/L yeast extract, 5 g/L Bacto Tryptone, 10 g/L sodium chloride) containing 100 μg/ml ampicillin contained in 125-mL Erlenmeyer flasks. Flasks were incubated for 16 hours at 37° C. in a rotary shaking incubator. After 16 hours, the cultures were diluted using fresh LB broth containing 100 μg/ml ampicillin to an optical density of 0.16 at 600 nm. 50 milliliters of the diluted cultures were placed in 300-mL Erlenmeyer flasks and incubated at 37° C. in a rotary shaking incubator until the optical density at 600 nm reached approximately 0.6, typically 90 minutes. Four milliliters of the cultures were then placed into 20-mL gas chromatography headspace vials. 2-methyl-3-buten-2-ol was added to a final concentration of 1 mM. IPTG (Isopropyl β-D-1-thiogalactopyranoside) was added to 1 mM. Cultures containing 2-methyl-3-buten-2-ol were grown for 16 hours at 37° C. with shaking.

Isoprene, 3-methyl-2-buten-1-ol and 2-methyl-3-buten-2-ol were measured as above. The identity of the isoprene peak was also verified using GC/MS, as described above.

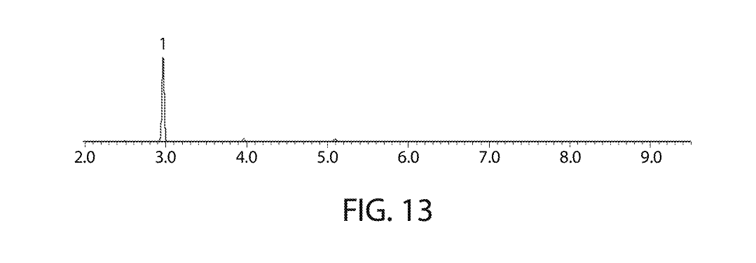

The results of this example are presented in FIG. 6 and FIG. 13. LB broth containing 1 mM 2-methyl-3-buten-2-ol with E. coli cells omitted showed a peak at 2.96 minutes corresponding to 2-methyl-3-buten-2-ol (FIG. 13). Similarly, cultures containing 1 mM 2-methyl-3-buten-2-ol and BL21(DE3) cells harboring pJ404-SAAT showed a peak at 2.96 minutes corresponding to 2-methyl-3-buten-2-ol (data not shown). In contrast, cultures containing 1 mM 2-methyl-3-buten-2-ol and BL21(DE3) cells harboring pJ404-LDI converted 2-methyl-3-buten-2-ol to isoprene, corresponding to the peak at 2.49 minutes (FIG. 6). This demonstrates that E. coli cells harboring pJ404-LDI dehydrate 3-methyl-2-buten-1-ol to isoprene.

### Example 4

### Microorganism for the Production of Isoprene from Glycerol

The following prophetic example demonstrates a non-naturally occurring microorganism engineered for the efficient conversion of glycerol to isoprene.

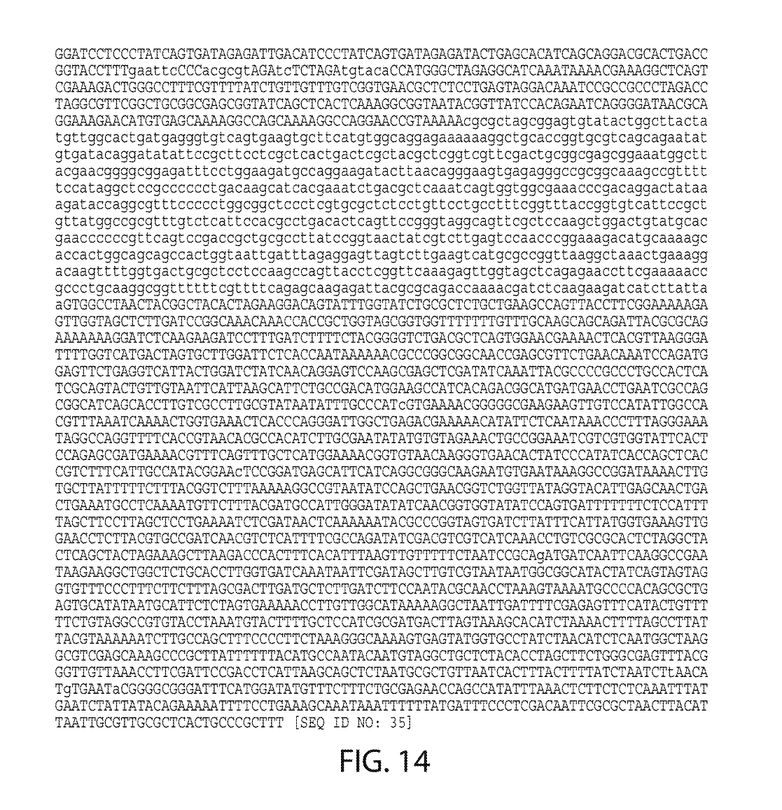

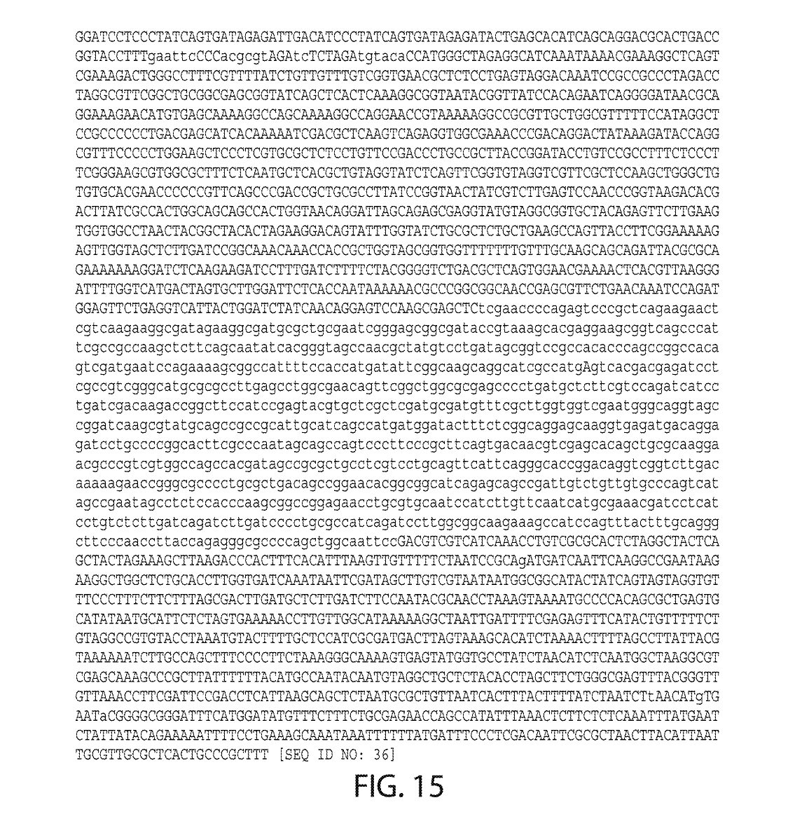

Plasmid pGA31R-MCS was constructed entirely by DNA synthesis by DNA2.0 (Menlo Park, Calif.), with the nucleotide sequence presented in FIG. 14. Plasmid pGE21R-MCS was constructed by replacing the chloramphenicol resistance marker and the p15A origin of replication of pGA31R-MCS with a kanamycin resistance marker and a ColE1 origin. The sequence of pGE21R-MCS is presented in FIG. 15.

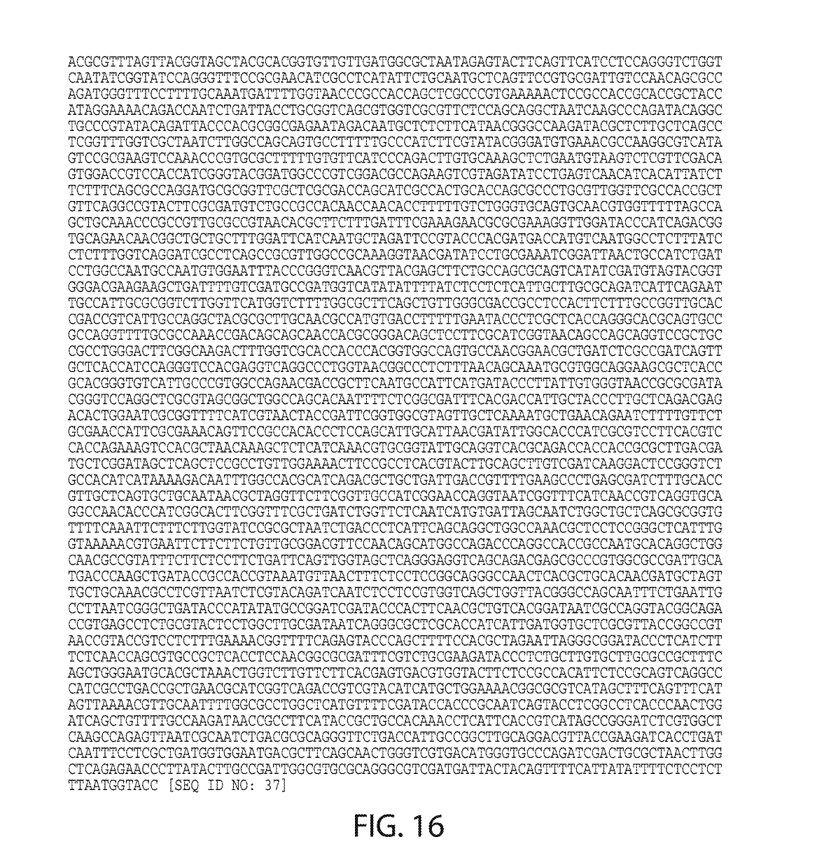

Plasmid pJ248-mvaES was constructed by DNA2.0 using the codon-optimized sequence of the mvaE and mvaS genes of Enterococcus faecalis ATCC 700802 (the codon-optimized sequences of mvaE and mvaS are as presented in FIG. 16). The mvaE and mvaS genes of Enterococcus faecalis ATCC 700802 were codon-optimized for expression in E. coli using the proprietary algorithms of DNA2.0, synthesized and inserted in the plasmid pJ248. Unique ribosomal binding sites were included in front of each gene, along with flanking endonuclease restriction sites for use in plasmid construction.

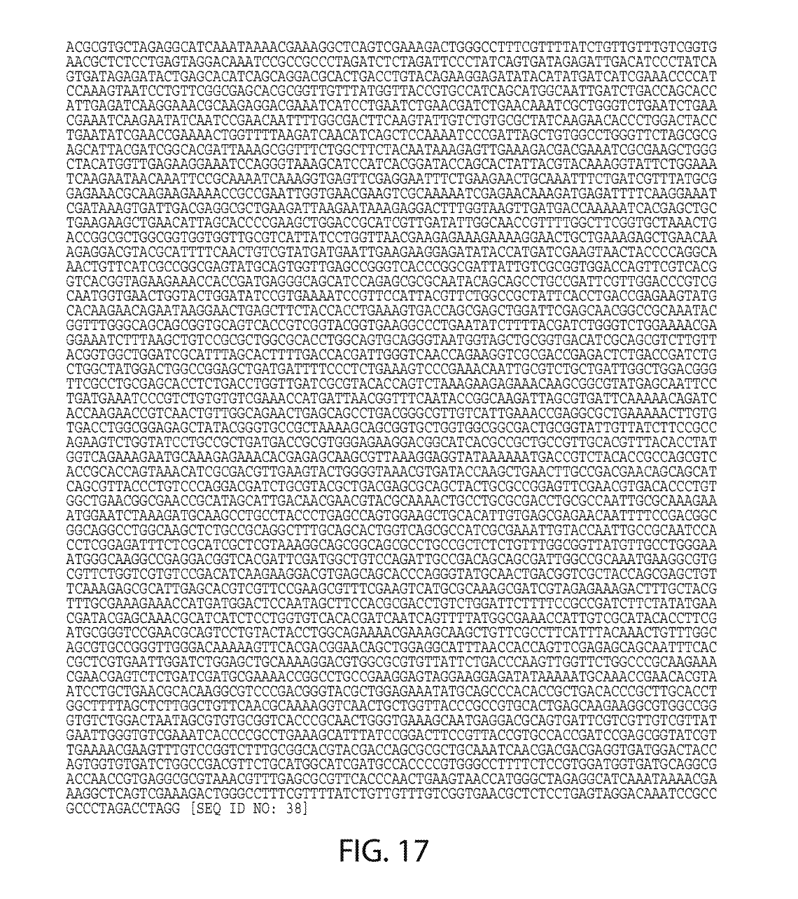

Plasmid pJ241-MK.PMK.MPD.IDI containing a codon-optimized synthetic operon was constructed entirely by DNA synthesis by DNA2.0, with the nucleotide sequence presented in FIG. 17. The sequence of the synthetic operon, codon-optimized for expression in E. coli, encodes the mevalonate kinase gene of Methanocaldococcus jannaschi, the phosphomevalonate kinase of Enterococcus faecalis ATCC 700802, the mevalonate pyrophosphate decarboxylase of Saccharomyces cerevisiae S288C, and the isopentenyl diphosphate isomerase gene of E. coli MG1655, including incorporated ribosomal binding sites and flanking restriction endonuclease sites used in subsequent cloning steps.

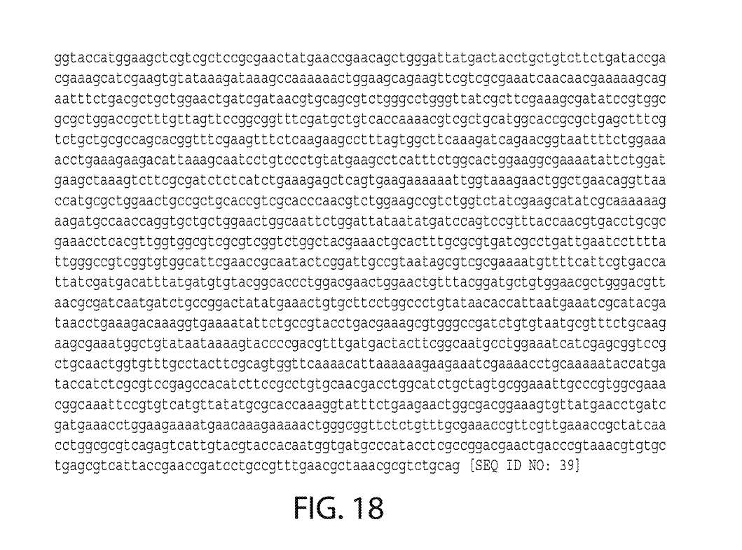

Plasmid pUC57-ispS was synthesized by Genscript (Piscataway, N.J.). The ispS coding region, with an associated ribosome binding site, is presented in FIG. 18.

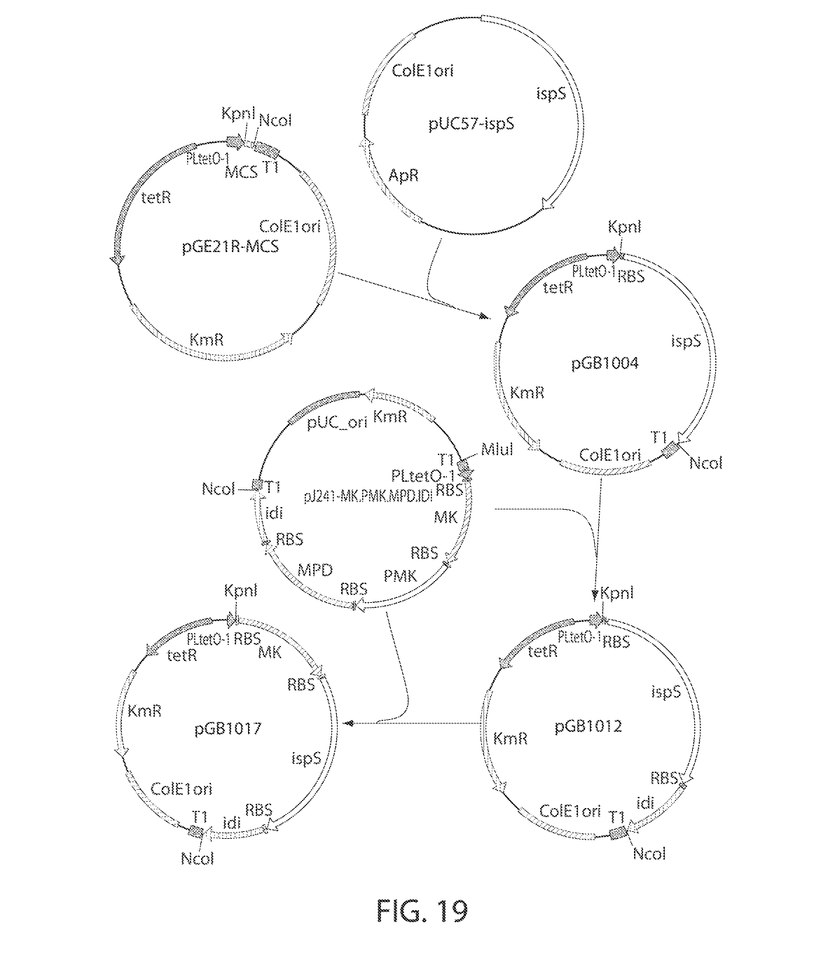

Plasmid pGB 1004 was constructed by inserting a PCR product encoding the codon-optimized ispS gene of P. alba into the KpnI and NcoI restriction endonuclease sites of plasmid pGE21R-MCS (FIG. 19). The PCR product encoding the ispS gene was amplified from the plasmid pUC57-ispS using AccuPrimer Pfx polymerase with the following oligonucleotide primers:

Primer 1:

[SEQ ID NO: 25]

5′-ATAGGTACCATTAAAGAGGAGAAAATATAATGGAAGCTCG
TCGCTCCG-3′
Primer 2:

[SEQ ID NO: 26]

5′-ATACCATGGACGTTTAGCGTTCAAACGGCAGG-3′

Primer 1 incorporates a ribosomal binding site in front of the start codon of ispS and a KpnI restriction site. Primer 2 incorporates an NcoI restriction site after the ispS stop codon. The PCR and plasmid pGE21R-MCS were digested with KpnI and NcoI, followed by gel purification. The fragments were cloned together using standard cloning techniques. FIG. 18 shows the codon-optimized sequence of the synthetic isoprene synthase gene of Populus alba, containing a ribosomal binding site and flanking restriction endonuclease sites used in subsequent cloning steps, but without the N-terminal transit peptide.

Plasmid pGB1012 was constructed by inserting a PCR product encoding the idi gene of E. coli into the NcoI site of pGB 1004 (FIG. 19). The PCR product encoding the idi gene was amplified from the plasmid pJ241-MK.PMK.MPD.IDI using AccuPrime Pfx polymerase with the following primers:

Primer 1:

SEQ ID NO: 27]

5′-CGCTAAACGTCCATGTAAGGAGATATAAAAATGCAAACCG-3′
Primer 2:

[SEQ ID NO: 28]

5′-TGATGCCTCTAGCCCATG-3′

Primer 1 maintains the ribosomal binding site in front of the start codon of idi. Primer 1 and Primer 2 also include appropriate vector-overlapping 5′ sequences for use with the In-Fusion Advantage PCR Cloning Kit (Clontech). The PCR product was gel-purified, as was pGB1004 linearized with the restriction endonuclease NcoI. Fragments were directionally joined together using the In-Fusion cloning kit and GC5 competent cells, following the manufacturer's directions. Transformants were screened, and the proper plasmid was identified through agarose gel electrophoresis of restriction endonuclease-digested plasmid DNAs.

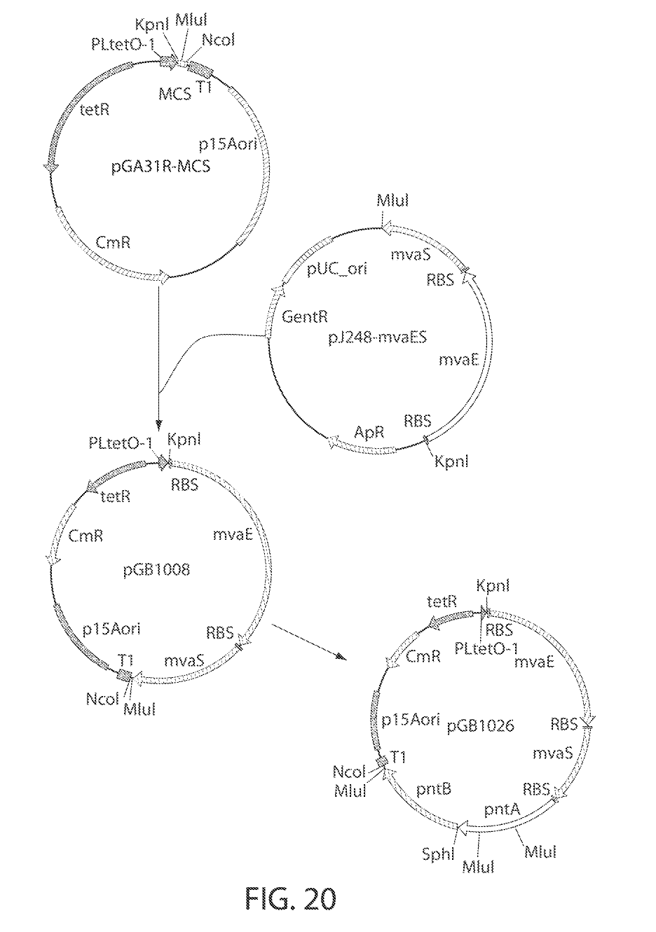

Plasmid pGB 1008 was constructed by cloning the optimized mvaES genes from pJ248-mvaES into pGA31R-MCS as a KpnI/MluI DNA fragment using standard cloning techniques (FIG. 20).

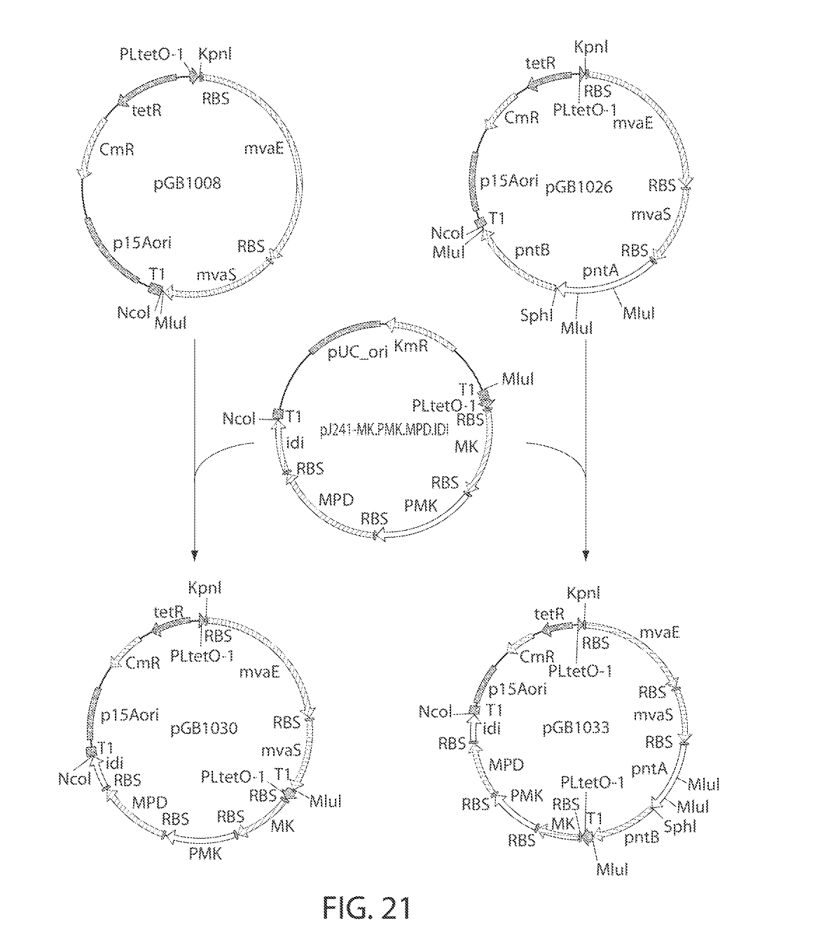

Plasmid pGB1030 was created through the following process (FIG. 21). pGB1008 was digested with the restriction endonucleases NcoI and MluI; the resulting 6.8 kb DNA fragment was gel-purified. Plasmid pJ241-MK.PMK.MPD.IDI was digested with the restriction endonucleases NcoI and MluI; the resulting 4.1 kb containing the synthetic operon was gel-purified. The fragments were ligated together using the NEB Quick Ligation Kit (New England Biolabs) and transformed into GC5 competent cells. Transformants were screened, and the proper plasmid was identified through agarose gel electrophoresis of restriction endonuclease-digested plasmid DNAs.

Plasmid pGB1030-GlyOH is created from plasmid pGB1030 and plasmid pZS-adhEp.glpK.glpD. A PCR fragment encoding a fragment of the E. coli adhE promoter, glpK, glpD, and the T1 terminator can be amplified from pZS-adhEp.glpK.glpD using Phusion Polymerase (NEB, Ipswich, Mass.) and the following primers:

Primer 1:

[SEQ ID NO: 29]

5′-TTTCGCCAGATATCGACGTCGGTTAGCTCCGAAGCAAAAGC-3′
Primer 2:

[SEQ ID NO: 30]

5′-ACAGGTTTGATGACGAACCCGTACCCTAGGTCTAGG-3′

The primers are designed to provide overlapping ends for use in an In-Fusion reaction, with the first primer remaking the AatII site. The second primer destroys the AatII site by replacing it with an AvrII site. The resulting 3.6 kB fragment is gel extracted and joined to pGB1030 fragment linearized with AatII using the IN-FUSION HD KIT™. The fragments are joined together using the IN-FUSION ADVANTAGE PCR CLONING KIT™ (Clontech Laboratories, Inc., Mountain View, Calif.), then transformed into chemically competent E. coli GC5 cells (Gene Choice, available from Sigma-Aldrich Co. LLC) following the manufacturer's directions. Transformants are screened, and the proper plasmid is identified through agarose gel electrophoresis of restriction endonuclease-digested plasmid DNAs.

Plasmids pGB1012 and pGB1030-G1y0H are co-transformed into MG1655 using electroporation, generating the strain herein referred to as MG1655(1012/1030-G1y0H). This combination of plasmids provides a mevalonate-based pathway for production of isoprene.

Seed cultures of MG1655(1012/1030-GlyOH) are prepared as follows: the cultures (stored as glycerol stocks at −80° C.) are used to inoculate 5 ml (LB medium as described above, containing appropriate amounts of chloramphenicol and kanamycin) seed cultures in 15 ml culture tubes and grown aerobically at 37° C. and 175 rpm for 16 hours. After 16 hours, the seed cultures are diluted into LB supplemented with appropriate antibiotics, 20 g/1 glycerol, and 100 μg/1 anhydrotetracycline to achieve an initial optical density of 0.3 at 600 nm. 10-ml aliquots of each diluted culture are placed in three 20-ml headspace vials; the diluted cultures are incubated at 37° C. and 175 rpm. At the end of 1, 2 and 3 hours of incubation, one of the headspace vials is removed from the shaking incubator, and the isoprene concentration is estimated by manually exposing a solid-phase microextraction fiber (85 gm Carboxen/PDMS) to sample the headspace. The fiber is desorbed at 300° C. for 30 seconds prior to insertion into the headspace vial, exposed in the vial at −37° C. for 60 seconds to extract the volatiles, and immediately desorbed in the injector of an Agilent 5890 Series II GC at 200° C. for 30 seconds (splitless injection, purge valve closed). The initial hold is at 30° C. for 5 minutes, followed by a ramp at 20° C./min to 230° C., with a final hold of 2 minutes. The carrier gas is helium, the FID detector temperature is kept at 250° C., and the column is a Rtx-5 (30 m×530 μm×3 μm). The samples are compared to a commercial isoprene standard. The concentration of isoprene in the samples may be calculated by comparison to calibration curves generated from diluted standards analyzed under the same GC/FID method.

### Example 5

### Microorganism for the Production of Isoprene From Dimethylallyl Diphosphate

This prophetic example demonstrates how one may produce isoprene with a non-naturally occurring microorganism expressing an MBO synthase, a 2-methyl-3-buten-2-ol isomerase, and a 2-methyl-3-buten-2-ol dehydratase.

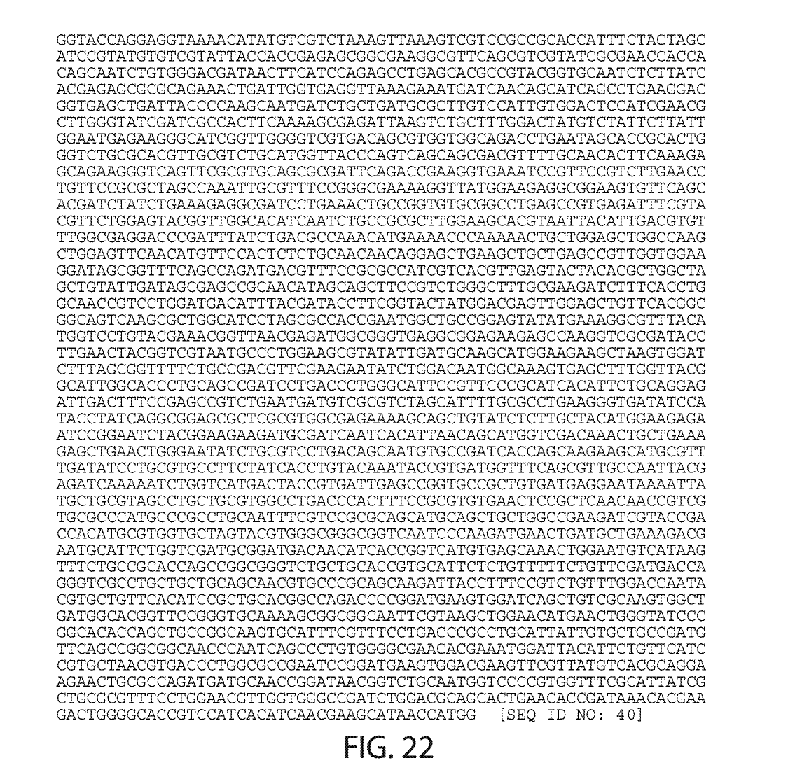

The Tps-MBOJ gene of Pinus sabiniana (GenBank Accession No. JF19039) and the idi gene of Haematococcus pluvialis are codon-optimized for expression in E. coli using the proprietary algorithms of DNA2.0 (including ribosome binding sites, a KpnI restriction site on the 5′ end and an NcoI restriction site on the 3′ end, FIG. 22), synthesized and inserted into the plasmid vector pJexpress40l to produce pJ401-MBO1.IDI.

The codon-optimized ldi gene may be PCR amplified from pJ404-LDI using Phusion Polymerase (NEB) and the following primers:

[SEQ ID NO: 31]

5′-CGAAGCATAACCATGTAGGAGGTAAAACATATGCACCACC-3′

[SEQ ID NO: 32]

5′-GCCCTTGGGGCCATGGTTACTTGCCCGCCAGCTTC-3′

pJ401-MBOLIDI is linearized by digestion with NcoI and purified by gel electrophoresis. The fragments are joined together using the IN-FUSION ADVANTAGE PCR CLONING KIT™ (Clontech Laboratories, Inc., Mountain View, Calif.), then transformed into chemically competent E. coli GC5 cells (GENE CHOICE™, available from Sigma-Aldrich Co. LLC) following the manufacturer's directions. Transformants are screened, and the proper plasmid is identified through agarose gel electrophoresis of restriction endonuclease-digested plasmid DNAs. The proper plasmid is then transformed into electrocompetent E. coli BL21(DE3). The resulting plasmid is designated pJ401-MBO1.IDI.LDI.

The production of isoprene by BL21(DE3) harboring plasmid pJ401-MBO1.IDI.LDI may be assayed as follows. A single colony of BL21(DE3) harboring pJ401-MBO1.IDI.LDI or pJ404-LDI from LB-agar plates are used to inoculate 10 milliliter aliquots of LB broth (10 g/L yeast extract, 5 g/L Bacto Tryptone, 10 g/L sodium chloride) containing 20 g/L glycerol and 100 μg/ml ampicillin contained in 125-mL Erlenmeyer flasks. Flasks are incubated for 16 hours at 37° C. in a rotary shaking incubator. After 16 hours, the cultures are diluted using fresh LB broth containing 20 g/L glycerol and 100 μg/ml ampicillin to an optical density of 0.16 at 600 nm. 50 milliliters of the diluted cultures are placed in 300-mL Erlenmeyer flasks and incubated at 37° C. in a rotary shaking incubator until the optical density at 600 nm reaches approximately 0.6, typically 90 minutes. Four milliliters of the cultures are then placed into 20-mL gas chromatography headspace vials. IPTG (Isopropyl (3-D-1-thiogalactopyranoside) is added to 1 mM. Cultures are grown for 16 hours at 37° C. with shaking.

Isoprene is measured using headspace analysis on an Agilent 7890A GC equipped with a CTC-PAL autosampler and a FID. Headspace vials (20 mL) are incubated at 50° C. with agitation at 500 rpm for 2 minutes. Then 1 mL of the headspace is removed using a heated headspace syringe at 50° C. and injected into the GC inlet (250° C., split of 20:1). Samples are analyzed using a FID detector set at 300° C., with a helium carrier gas flow rate of 2 ml/min through a DB-624 30 m×530 μm×3 μm column (J&W Scientific), and an oven program of 85° C. for 5.25 minutes. The isoprene concentration in samples is calculated from calibration curves generated from isoprene calibration gas standards analyzed under the same GC/FID method. The isoprene product is also confirmed by headspace GC/MS using an Agilent 7890A GC equipped with a 5975C MSD and a CTC-PAL autosampler. Headspace vials are incubated at 85° C. with agitation at 600 rpm for 5 minutes. Then 1 mL of the headspace is removed using a heated headspace syringe at 85° C. and injected into the GC inlet (250° C., split of 25:1). The GC/MS method uses helium as the carrier gas at 1 mL/min through a HP-5MS 30 m x 250 μm x 0.25 μm column (J&W Scientific), an oven program of 35° C. for 4 minutes, then ramped 25° C./min to 150° C., a MS source temperature of 230° C., and a quadrupole temperature of 150° C. The mass spectrometer is operated in scan mode from 25 to 160 mass units. The isoprene peak is identified by the NIST 11 MS Library, as well as comparison against an authentic sample (135 ppm isoprene, 135 ppm carbon dioxide in dry nitrogen gas, Matheson TRIGAS, Houston, Tex.).

2-methyl-3-buten-1-ol is measured using headspace analysis on an Agilent 7890A GC equipped with a CTC-PAL autosampler and a FID. Headspace vials (20 mL) are incubated at 85° C. with agitation at 600 rpm for 5 minutes. Then 1 mL of the headspace is removed using a heated headspace syringe at 85° C. and injected into the GC inlet (250° C., split of 25:1). Samples are analyzed using a FID detector set at 350° C., with a helium carrier gas flow rate of 3 ml/min through at DB-624 30 m×530 μm×3 μm column (J&W Scientific), and an oven program of 90° C., then ramping 20° C./min to 230° C. for 3 minutes. The 2-methyl-3-buten-2-ol concentration in samples is calculated from calibration curves generated from diluted standards of the compound analyzed under the same GC/FID method.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While the foregoing is directed to embodiments of the present invention, other and further embodiments of the invention may be devised without departing from the basic scope thereof, and the scope thereof is determined by the claims that follow.

What is claimed is:

1. A non-naturally occurring microorganism comprising:
a glycerol dissimilation pathway comprising a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and dihydroxyacetone kinase are encoded by an exogenous nucleic acid; and
an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, a 2-methyl-3-buten-2-ol synthase, and a 2-methyl-3-buten-2-ol dehydratase, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase, 2-methyl-3-buten-2-ol synthase, and 2-methyl-3-buten-2-ol dehydratase are encoded by an exogenous nucleic acid,
wherein the glycerol dissimilation pathway and the isoprene pathway are expressed in sufficient amounts to produce isoprene from glycerol.

2. The non-naturally occurring microorganism according to claim 1 wherein the microorganism further comprises an exogenous nucleic acid encoding a glycerol facilitator protein in sufficient amounts to improve the rate of glycerol dissimilation as compared to a parental strain without the exogenous nucleic acid.

3. The non-naturally occurring microorganism according to claim 1 wherein the microorganism has been modified to have reduced or eliminated activity of one or more of the following enzymes: lactate dehydrogenase, succinate dehydrogenase, acetate kinase, phosphate acetyltransferase, pyruvate oxidase, and alcohol dehydrogenase.

4. A non-naturally occurring microorganism comprising:
a glycerol dissimilation pathway comprising a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and dihydroxyacetone kinase are encoded by an exogenous nucleic acid; and
an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, a 3-methyl-2-buten-1-ol synthase, a 2-methyl-3-buten-2-ol isomerase, and a 2-methyl-3-buten-2-ol dehydratase, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase, 3-methyl-2-buten-1-ol synthase, 2-methyl-3-buten-2-ol isomerase, and 2-methyl-3-buten-2-ol dehydratase are encoded by an exogenous nucleic acid,
wherein the glycerol dissimilation pathway and the isoprene pathway are expressed in sufficient amounts to produce isoprene from glycerol.

5. The non-naturally occurring microorganism according to claim 4 wherein the microorganism further comprises an exogenous nucleic acid encoding a glycerol facilitator protein in sufficient amounts to improve the rate of glycerol dissimilation as compared to a parental strain without the exogenous nucleic acid.

6. The non-naturally occurring microorganism according to claim 4 wherein the microorganism has been modified to have reduced or eliminated activity of one or more of the following enzymes: lactate dehydrogenase, succinate dehydrogenase, acetate kinase, phosphate acetyltransferase, pyruvate oxidase, and alcohol dehydrogenase.

7. A non-naturally occurring microorganism comprising:
a glycerol dissimilation pathway comprising a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and dihydroxyacetone kinase are encoded by an exogenous nucleic acid; and
an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, and a monoterpene synthase, sesquiterpene synthase or diterpene synthase capable of converting dimethylallyl diphosphate into isoprene, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase and monoterpene synthase, sesquiterpene synthase or diterpene synthase are encoded by an exogenous nucleic acid,
wherein the glycerol dissimilation pathway and the isoprene pathway are expressed in sufficient amounts to produce isoprene from glycerol.

8. The non-naturally occurring microorganism according to claim 7 wherein the monoterpene synthase, sesquiterpene synthase, or diterpene synthase is a myrcene, ocimene, or farnesene synthase with altered substrate specificity.

9. The non-naturally occurring microorganism according to claim 7 wherein the microorganism further comprises an exogenous nucleic acid encoding a glycerol facilitator protein in sufficient amounts to improve the rate of glycerol dissimilation as compared to a parental strain without the exogenous nucleic acid.

10. The non-naturally occurring microorganism according to claim 7 wherein the microorganism has been modified to have reduced or eliminated activity of one or more of the following enzymes: lactate dehydrogenase, succinate dehydrogenase, acetate kinase, phosphate acetyltransferase, pyruvate oxidase, and alcohol dehydrogenase.

11. A non-naturally occurring microorganism comprising:
a glycerol dissimilation pathway comprising a glycerol kinase and a glycerol-3-phosphate dehydrogenase or a glycerol dehydrogenase and a dihydroxyacetone kinase, wherein one or more of the glycerol kinase, glycerol-3-phosphate dehydrogenase, glycerol dehydrogenase, and dihydroxyacetone kinase are encoded by an exogenous nucleic acid; and
an isoprene pathway comprising an acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, a isopentenyl diphosphate isomerase, and an isoprene synthase, wherein one or more of the acetyl-CoA C-acetyltransferase, hydroxymethylglutaryl-CoA synthase, hydroxymethylglutaryl-CoA reductase, mevalonate kinase, phosphomevalonate kinase, diphosphomevalonate decarboxylase, isopentenyl diphosphate isomerase and isoprene synthase are encoded by an exogenous nucleic acid,
wherein the glycerol dissimilation pathway and the isoprene pathway are expressed in sufficient amounts to produce isoprene from glycerol.

12. The non-naturally occurring microorganism according to claim 11 wherein the microorganism further comprises an exogenous nucleic acid encoding a glycerol facilitator protein in sufficient amounts to improve the rate of glycerol dissimilation as compared to a parental strain without the exogenous nucleic acid.

13. The non-naturally occurring microorganism according to claim 11 wherein the microorganism has been modified to have reduced or eliminated activity of one or more of the following enzymes: lactate dehydrogenase, succinate dehydrogenase, acetate kinase, phosphate acetyltransferase, pyruvate oxidase, and alcohol dehydrogenase.

14. The non-naturally occurring microorganism according to claim 11 wherein the isoprene synthase is an isoprene synthase from Populus alba, Robinia pseudoacacia or the genus Wisteria.

15-28. (canceled)

29. A method for producing isoprene comprising culturing the non-naturally occurring microorganism of claim 1 under suitable conditions in a medium comprising glycerol for a sufficient period of time to produce isoprene from the glycerol and recovering the isoprene.

30. A method for producing isoprene comprising culturing the non-naturally occurring microorganism of claim 4 under suitable conditions in a medium comprising glycerol for a sufficient period of time to produce isoprene from the glycerol and recovering the isoprene.

31. A method for producing isoprene comprising culturing the non-naturally occurring microorganism of claim 7 under suitable conditions in a medium comprising glycerol for a sufficient period of time to produce isoprene from the glycerol and recovering the isoprene.

32. A method for producing isoprene comprising culturing the non-naturally occurring microorganism of claim 11 under suitable conditions in a medium comprising glycerol for a sufficient period of time to produce isoprene from the glycerol and recovering the isoprene.


SEQUENCE LISTING (41 sequences)

SEQ ID NO: 1 - DNA, 29 bp, Artificial Sequence
  Description of Artificial Sequence Synthetic primer
atcgagctcaaccatcatcgatgaattgc

SEQ ID NO: 2 - DNA, 25 bp, Artificial Sequence
  Description of Artificial Sequence Synthetic primer
tctggtaccctgaagcttgcatgcc

SEQ ID NO: 3 - DNA, 23 bp, Artificial Sequence
  Description of Artificial Sequence Synthetic primer
atagagctcccaccagataacgg

SEQ ID NO: 4 - DNA, 27 bp, Artificial Sequence
  Description of Artificial Sequence Synthetic primer
gcgctcgagtttcataagactttctcc

SEQ ID NO: 5 - DNA, 26 bp, Artificial Sequence
  Description of Artificial Sequence Synthetic primer
gacctcgagctggtttaatcttgccg

SEQ ID NO: 6 - DNA, 25 bp, Artificial Sequence
  Description of Artificial Sequence Synthetic primer
cgtggtaccttctttcagcatttcg

SEQ ID NO: 7 - DNA, 22 bp, Artificial Sequence
  Description of Artificial Sequence Synthetic primer
attgagctcggttaccgtcgcc

SEQ ID NO: 8 - DNA, 32 bp, Artificial Sequence
  Description of Artificial Sequence Synthetic primer
gccattcgccttctcgagcacgacattcctcc

SEQ ID NO: 9 - DNA, 32 bp, Artificial Sequence
  Description of Artificial Sequence Synthetic primer
ggaggaatctcgtgctcgagaaggcgaatggc

SEQ ID NO: 10 - DNA, 24 bp, Artificial Sequence
  Description of Artificial Sequence Synthetic primer
agcggtaccacagttgatgcaacc

SEQ ID NO: 11 - DNA, 22 bp, Artificial Sequence
  Description of Artificial Sequence Synthetic primer
attgagctcggttaccgtcgcc

SEQ ID NO: 12 - DNA, 24 bp, Artificial Sequence
  Description of Artificial Sequence Synthetic primer
agcggtaccacagttgatgcaacc

SEQ ID NO: 13 - DNA, 25 bp, Artificial Sequence
  Description of Artificial Sequence Synthetic primer
acggagctcatcttgataacgcgat

SEQ ID NO: 14 - DNA, 29 bp, Artificial Sequence
  Description of Artificial Sequence Synthetic primer
tacctcgagcatggaagtacctataattg

SEQ ID NO: 15 - DNA, 35 bp, Artificial Sequence
  Description of Artificial Sequence Synthetic primer
ggataaaccgtgtccctcgagcagcagtaatctcg

SEQ ID NO: 16 - DNA, 28 bp, Artificial Sequence
  Description of Artificial Sequence Synthetic primer
actggtacctctagaagttcatcaggcc

SEQ ID NO: 17 - DNA, 28 bp, Artificial Sequence
  Description of Artificial Sequence Synthetic primer
atagagctcagcgaacaatgcaatagcc

SEQ ID NO: 18 - DNA, 46 bp, Artificial Sequence
  Description of Artificial Sequence Synthetic primer
cagcgctactgattaagctgtacaagtaacagccataatgctctcc

SEQ ID NO: 19 - DNA, 46 bp, Artificial Sequence
  Description of Artificial Sequence Synthetic primer
ggagagcattatggctgttacttgtacagcttaatcagtagcgctg

SEQ ID NO: 20 - DNA, 27 bp, Artificial Sequence
  Description of Artificial Sequence Synthetic primer
acagcatgcgaacaccacttccagagc

SEQ ID NO: 21 - DNA, 28 bp, Artificial Sequence
  Description of Artificial Sequence Synthetic primer
atagagctcagcgaacaatgcaatagcc

SEQ ID NO: 22 - DNA, 27 bp, Artificial Sequence
  Description of Artificial Sequence Synthetic primer
acagcatgcgaacaccacttccagagc

SEQ ID NO: 23 - DNA, 30 bp, Artificial Sequence
  Description of Artificial Sequence Synthetic primer
cgcctcgagggttagctccgaagcaaaagc

SEQ ID NO: 24 - DNA, 33 bp, Artificial Sequence
  Description of Artificial Sequence Synthetic primer
cgcggtaccaatgctctcctgataatgttaaac

SEQ ID NO: 25 - DNA, 48 bp, Artificial Sequence
  Description of Artificial Sequence Synthetic primer
ataggtaccattaaagaggagaaaatataatggaagctcgtcgctccg

SEQ ID NO: 26 - DNA, 32 bp, Artificial Sequence
  Description of Artificial Sequence Synthetic primer
ataccatggacgtttagcgttcaaacggcagg

SEQ ID NO: 27 - DNA, 40 bp, Artificial Sequence
  Description of Artificial Sequence Synthetic primer
cgctaaacgtccatgtaaggagatataaaaatgcaaaccg

SEQ ID NO: 28 - DNA, 18 bp, Artificial Sequence
  Description of Artificial Sequence Synthetic primer
tgatgcctctagcccatg

SEQ ID NO: 29 - DNA, 41 bp, Artificial Sequence
  Description of Artificial Sequence Synthetic primer
tttcgccagatatcgacgtcggttagctccgaagcaaaagc

SEQ ID NO: 30 - DNA, 36 bp, Artificial Sequence
  Description of Artificial Sequence Synthetic primer
acaggtttgatgacgaacccgtaccctaggtctagg

SEQ ID NO: 31 - DNA, 40 bp, Artificial Sequence
  Description of Artificial Sequence Synthetic primer
cgaagcataaccatgtaggaggtaaaacatatgcaccacc

SEQ ID NO: 32 - DNA, 35 bp, Artificial Sequence
  Description of Artificial Sequence Synthetic primer
gcccttggggccatggttacttgcccgccagcttc

SEQ ID NO: 33 - DNA, 1235 bp, Artificial Sequence
  Description of Artificial Sequence Synthetic polynucleotide
aggaggtaaaacatatgcaccaccatcatcaccacatgcgctttacgttgaaaaccaccgccatcgtgtccgctgcggcgttgctggcaggtttcggtccgccaccgcgtgcggcggagctgccgccaggccgtctggcgaccaccgaagattacttcgcgcaacaagcgaaacaggcggtgacgccggatgtgatggcacaactggcctacatgaactacattgacttcatcagcccgttctacagccgtggttgcagctttgaagcgtgggagttgaagcatacgccgcaacgcgtcattaagtatagcattgcgttctatgcatacggtctggcgtcggtcgcactgattgacccgaagctgcgtgccctggcaggtcacgatctggatatcgcggtgtctaaaatgaagtgcaagcgtgtttggggtgactgggaagaggatggttttggcaccgacccgatcgagaaagagaacatcatgtacaaaggtcatctgaacctgatgtatggcctgtatcagctggtgacgggtagccgtcgctacgaggcagagcacgcgcacctgacccgtatcatccatgacgagattgccgctaatccgttcgccggcatcgtatgtgaaccggacaattactttgtccagtgtaacagcgtcgcgtacttgagcctgtgggtttatgaccgtctgcacggcactgattatcgcgcagccacccgtgcttggttggatttcattcagaaggacctgatcgacccggagcgcggtgcattctacctgtcttaccacccggaaagcggtgctgttaagccgtggattagcgcgtataccactgcatggacgctggccatggttcacggcatggatccggcgtttagcgagcgctactatccgcgcttcaaacagaccttcgttgaagtgtacgacgagggccgtaaagcccgggttcgtgaaaccgccggtaccgacgacgccgacggtggcgtgggtctggcgagcgcgtttacgctgttgttggcacgtgagatgggcgatcagcaactgtttgatcagctgctgaatcatctggaaccgcctgccaaaccgagcattgtcagcgcgtccctgcgttatgaacaccctggctccctgctgtttgatgagctgctgttcctggctaaagttcatgcaggttttggtgcgctgctgcgtatgccgccaccggcagcgaagctggcgggcaagtaactcgag

SEQ ID NO: 34 - DNA, 1350 bp, Artificial Sequence
  Description of Artificial Sequence Synthetic polynucleotide
aggaggtaaaacatatgcatcatcaccaccaccacatggaaaagatcgaggtaagcattaacagcaaacatacgattaaaccgagcacgagctccaccccgttgcagccgtataagctgaccctgctggaccaactgaccccaccggcatacgtcccaatcgtctttttctatccgattacggatcacgacttcaatctgccgcagacgctggcagacctgcgtcaagccctgtccgaaaccctgactctgtattacccgctgagcggtcgtgtgaagaataacttgtacattgacgacttcgaagagggcgttccgtacctggaagcgcgtgtcaattgtgatatgacggacttcctgcgcctgcgtaagattgagtgtttgaacgagttcgtgccgattaagccgtttagcatggaagccattagcgacgagcgttacccgttgctgggtgttcaagtcaacgtgttcgatagcggtatcgcgatcggtgtttcggtttctcataaactgatcgacggcggcaccgcggactgcttcctgaaatcctggggtgcggttttccgcggttgccgcgagaatatcatccacccgagcctgagcgaggcggcactgctgttcccaccgcgcgatgatttgccggagaaatatgttgatcagatggaagccctgtggttcgcgggtaaaaaggttgcgacccgtcgctttgtctttggtgttaaggcgatcagcagcatccaggacgaggcaaagtctgaatcggtgcctaagccgtcccgtgtgcacgcggtcaccggctttctgtggaagcacctgattgcggcaagccgtgctctgacctctggcaccacctcgacgcgcctgagcattgcggcacaggccgttaatctgcgtacccgcatgaacatggaaactgtgctggacaatgcgaccggcaacctgttttggtgggcgcaggctattctggagttgagccacaccaccccggagatcagcgatctgaagctgtgcgatctggtgaacttgttgaatggcagcgttaaacaatgcaatggtgattacttcgaaacgtttaaaggtaaagagggctatggccgtatgtgtgaatatctggattttcagcgtacgatgagcagcatggagccggcaccggatatctacctgtttagcagctggacgaacttctttaacccgctggactttggttggggtcgtaccagctggatcggtgtcgcaggtaagatcgagagcgccagctgcaaattcattattctggtgcctacccaatgtggctctggtatcgaggcttgggtgaacctggaagaagagaaaatggccatgctggaacaag

SEQ ID NO: 35 - DNA, 3228 bp, Artificial Sequence
  Description of Artificial Sequence Synthetic polynucleotide
ggatcctccctatcagtgatagagattgacatccctatcagtgatagagatactgagcacatcagcaggacgcactgaccggtacctttgaattccccacgcgtagatctctagatgtacaccatgggctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaacgcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatcgtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtcgtcatcaaacctgtcgcgcactctaggctactcagctactagaaagcttaagacccactttcacatttaagttgtttttctaatccgcagatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagccttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatcttaacatgtgaatacggggcgggatttcatggatatgtttctttctgcgagaaccagccatatttaaactcttctctcaaatttatgaatctattatacagaaaaattttcctgaaagcaaataaattttttatgatttccctcgacaattcgcgctaacttacattaattgcgttgcgctcactgcccgcttt

SEQ ID NO: 36 - DNA, 2984 bp, Artificial Sequence
  Description of Artificial Sequence Synthetic polynucleotide
ggatcctccctatcagtgatagagattgacatccctatcagtgatagagatactgagcacatcagcaggacgcactgaccggtacctttgaattccccacgcgtagatctctagatgtacaccatgggctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctctcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccgacgtcgtcatcaaacctgtcgcgcactctaggctactcagctactagaaagcttaagacccactttcacatttaagttgtttttctaatccgcagatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagccttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatcttaacatgtgaatacggggcgggatttcatggatatgtttctttctgcgagaaccagccatatttaaactcttctctcaaatttatgaatctattatacagaaaaattttcctgaaagcaaataaattttttatgatttccctcgacaattcgcgctaacttacattaattgcgttgcgctcactgcccgcttt

SEQ ID NO: 37 - DNA, 3611 bp, Artificial Sequence
  Description of Artificial Sequence Synthetic polynucleotide
acgcgtttagttacggtagctacgcacggtgttgttgatggcgctaatagagtacttcagttcatcctccagggtctggtcaatatcggtatccagggtttccgcgaacatcgcctcatattctgcaatgctcagttccgtgcgattgtccaacagcgccagatgggtttccttttgcaaatgattttggtaacccgccaccagctcgcccgtgaaaaactccgccaccgcaccgctaccataggaaaacagaccaatctgattacctgcggtcagcgtggtcgcgttctccagcaggctaatcaagcccagatacaggctgcccgtatacagattacccacgcggcgagaatagacaatgctctcttcataacgggccaagatacgctcttgctcagcctcggtttggtcgctaatcttggccagcagtgcctttttgcccatcttcgtatacgggatgtgaaacgccaaggcgtcatagtccgcgaagtccaaacccgtgcgctttttgtgttcatcccagacttgtgcaaagctctgaatgtaagtctcgttcgacagtggaccgtccaccatcgggtacggatggcccgtcggacgccagaagtcgtagatatcctgagtcaacatcacattatcttctttcagcgccaggatgcgcggttcgctcgcgaccagcatcgccactgcaccagcgccctgcgttggttcgccaccgctgttcaggccgtacttcgcgatgtctgccgccacaaccaacacctttttgtctgggtgcagtgcaacgtggtttttagccagctgcaaacccgccgttgcgccgtaacacgcttctttgatttcgaaagaacgcgcgaaaggttggatacccatcagacggtgcagaacaacggctgctgctttggattcatcaatgctagattccgtacccacgatgaccatgtcaatggcctctttatcctctttggtcaggatcgcctcagccgcgttggccgcaaaggtaacgatatcctgcgaaatcggattaactgccatctgatcctggccaatgccaatgtggaatttacccgggtcaacgttacgagcttctgccagcgcagtcatatcgatgtagtacggtgggacgaagaagctgattttgtcgatgccgatggtcatatattttatctcctctcattgcttgcgcagatcattcagaattgccattgcgcggtcttggttcatggtcttttggcgcttcagctgttgggcgaccgcctccacttctttgccggttgcaccgaccgtcattgccaggctacgcgcttgcaacgccatgtgacctttttgaataccctcgctcaccagggcacgcagtgccgccaggttttgcgccaaaccgacagcagcaaccacgcgggacagctccttcgcatcggtaacagccagcaggtccgctgccgcctgggacttcggcaagactttggtcgcaccacccacggtggccagtgccaacggaacgctgatctcgccgatcagttgctcaccatccagggtccacgaggtcaggccctggtaacggccctctttaacagcaaatgcgtggcaggaagcgctcaccgcacgggtgtcattgcccgtggccagaacgaccgcttcaatgccattcatgatacccttattgtgggtaaccgcgcgatacgggtccaggctcgcgtagcggctggccagcacaattttctcggcgatttcacgaccattgctacccttgctcagacgagacactggaatcgcggttttcatcgtaactaccgattcggtggcgtagttgctcaaaatgctgaacagaatcttttgttctgcgaaccattcgcgaaacagttccgccacaccctccagcattgcattaacgatattggcacccatcgcgtccttcacgtccaccagaaagtccacgctaacaaagctctcatcaaacgtgcggtattgcaggtcacgcagaccaccaccgcgcttgacgatgctcggatagctcagctccgcctgttggaaaacttccgcctcacgtacttgcagcttgtcgatcaaggactccgggtctgccacatcataaaagacaatttggccacgcatcagacgctgctgattgaccgttttgaagccctgagcgatctttgcaccgttgctcagtgctgcaataacgctaggttcttcggttgccatcggaaccaggtaatcggtttcatcaaccgtcaggtgcaggccaacacccatcggcacttcggtttcgctgatctggttctcaatcatgtgattagcaatctggctgctcagcgcggtgttttcaaattctttcttggtatccgcgctaatctgaccctcattcagcaggctggccaaacgctcctccgggctcatttggtaaaaacgtgaattcttcttctgttgcggacgttccaacagcatggccagacccaggccaccgccaatgcacaggctggcaacgccgtatttcttctccttctgattcagttggtagctcagggaggtcagcagacgagcgcccgtggcgccgattgcatgacccaagctgataccgccaccgtaaatgttaactttctcctccggcagggccaactcacgctgcacaacgatgctagttgctgcaaacgcctcgttaatctcgtacagatcaatctcctccgtggtcagctggttacgggccagcaatttctgaattgccttaatcgggctgatacccatatatgccggatcgatacccacttcaacgctgtcacggataatcgccaggtacggcagaccgtgagcctctgcgtactcctggcttgcgataatcagggcgctcgcaccatcattgatggtgctcgcgttaccggccgtaaccgtaccgtcctctttgaaaacggttttcagagtacccagcttttccacgctagaattagggcggataccctcatctttctcaaccagcgtgccgctcacctccaacggcgcgatttcgtctgcgaagataccctctgcttgtgcttgcgccgctttcagctgggaatgcacgctaaactggtcttgttcttcacgagtgacgtggtacttctccgccacattctccgcagtcaggcccatcgcctgaccgctgaacgcatcggtcagaccgtcgtacatcatgctggaaaacggcgcgtcatagctttcagtttcatagttaaaacgttgcaattttggcgcctggctcatgttttcgataccacccgcaatcagtacctcggcctcacccaactggatcagctgttttgccaagataaccgccttcataccgctgccacaaacctcattcaccgtcatagccgggatctcgtggctcaagccagagttaatcgcaatctgacgcgcagggttctgaccattgccggcttgcaggacgttaccgaagatcacctgatcaatttcctcgctgatggtggaatgacgcttcagcaactgggtcgtgacatgggtgcccagatcgactgcgctaacttggctcagagaacccttatacttgccgattggcgtgcgcagggcgtcgatgattactacagttttcattatattttctcctctttaatggtacc

SEQ ID NO: 38 - DNA, 4174 bp, Artificial Sequence
  Description of Artificial Sequence Synthetic polynucleotide
acgcgtgctagaggcatcaaataaaacgaaaggctcagtcgaaagactgggcctttcgttttatctgttgtttgtcggtgaacgctctcctgagtaggacaaatccgccgccctagatctctagattccctatcagtgatagagattgacatccctatcagtgatagagatactgagcacatcagcaggacgcactgacctgtacagaaggagatatacatatgatcatcgaaaccccatccaaagtaatcctgttcggcgagcacgcggttgtttatggttaccgtgccatcagcatggcaattgatctgaccagcaccattgagatcaaggaaacgcaagaggacgaaatcatcctgaatctgaacgatctgaacaaatcgctgggtctgaatctgaacgaaatcaagaatatcaatccgaacaattttggcgacttcaagtattgtctgtgcgctatcaagaacaccctggactacctgaatatcgaaccgaaaactggttttaagatcaacatcagctccaaaatcccgattagctgtggcctgggttctagcgcgagcattacgatcggcacgattaaagcggtttctggcttctacaataaagagttgaaagacgacgaaatcgcgaagctgggctacatggttgagaaggaaatccagggtaaagcatccatcacggataccagcactattacgtacaaaggtattctggaaatcaagaataacaaattccgcaaaatcaaaggtgagttcgaggaatttctgaagaactgcaaatttctgatcgtttatgcggagaaacgcaagaagaaaaccgccgaattggtgaacgaagtcgcaaaaatcgagaacaaagatgagattttcaaggaaatcgataaagtgattgacgaggcgctgaagattaagaataaagaggactttggtaagttgatgaccaaaaatcacgagctgctgaagaagctgaacattagcaccccgaagctggaccgcatcgttgatattggcaaccgttttggcttcggtgctaaactgaccggcgctggcggtggtggttgcgtcattatcctggttaacgaagagaaagaaaaggaactgctgaaagagctgaacaaagaggacgtacgcattttcaactgtcgtatgatgaattgaagaaggagatataccatgatcgaagtaactaccccaggcaaactgttcatcgccggcgagtatgcagtggttgagccgggtcacccggcgattattgtcgcggtggaccagttcgtcacggtcacggtagaagaaaccaccgatgagggcagcatccagagcgcgcaatacagcagcctgccgattcgttggacccgtcgcaatggtgaactggtactggatatccgtgaaaatccgttccattacgttctggccgctattcacctgaccgagaagtatgcacaagaacagaataaggaactgagcttctaccacctgaaagtgaccagcgagctggattcgagcaacggccgcaaatacggtttgggcagcagcggtgcagtcaccgtcggtacggtgaaggccctgaatatcttttacgatctgggtctggaaaacgaggaaatctttaagctgtccgcgctggcgcacctggcagtgcagggtaatggtagctgcggtgacatcgcagcgtcttgttacggtggctggatcgcatttagcacttttgaccacgattgggtcaaccagaaggtcgcgaccgagactctgaccgatctgctggctatggactggccggagctgatgattttccctctgaaagtcccgaaacaattgcgtctgctgattggctggacgggttcgcctgcgagcacctctgacctggttgatcgcgtacaccagtctaaagaagagaaacaagcggcgtatgagcaattcctgatgaaatcccgtctgtgtgtcgaaaccatgattaacggtttcaataccggcaagattagcgtgattcaaaaacagatcaccaagaaccgtcaactgttggcagaactgagcagcctgacgggcgttgtcattgaaaccgaggcgctgaaaaacttgtgtgacctggcggagagctatacgggtgccgctaaaagcagcggtgctggtggcggcgactgcggtattgttatcttccgccagaagtctggtatcctgccgctgatgaccgcgtgggagaaggacggcatcacgccgctgccgttgcacgtttacacctatggtcagaaagaatgcaaagagaaacacgagagcaagcgttaaaggaggtataaaaaatgaccgtctacaccgccagcgtcaccgcaccagtaaacatcgcgacgttgaagtactggggtaaacgtgataccaagctgaacttgccgacgaacagcagcatcagcgttaccctgtcccaggacgatctgcgtacgctgacgagcgcagctactgcgccggagttcgaacgtgacaccctgtggctgaacggcgaaccgcatagcattgacaacgaacgtacgcaaaactgcctgcgcgacctgcgccaattgcgcaaagaaatggaatctaaagatgcaagcctgcctaccctgagccagtggaagctgcacattgtgagcgagaacaattttccgacggcggcaggcctggcaagctctgccgcaggctttgcagcactggtcagcgccatcgcgaaattgtaccaattgccgcaatccacctcggagatttctcgcatcgctcgtaaaggcagcggcagcgcctgccgctctctgtttggcggttatgttgcctgggaaatgggcaaggccgaggacggtcacgattcgatggctgtccagattgccgacagcagcgattggccgcaaatgaaggcgtgcgttctggtcgtgtccgacatcaagaaggacgtgagcagcacccagggtatgcaactgacggtcgctaccagcgagctgttcaaagagcgcattgagcacgtcgttccgaagcgtttcgaagtcatgcgcaaagcgatcgtagagaaagactttgctacgtttgcgaaagaaaccatgatggactccaatagcttccacgcgacctgtctggattcttttccgccgatcttctatatgaacgatacgagcaaacgcatcatctcctggtgtcacacgatcaatcagttttatggcgaaaccattgtcgcatacaccttcgatgcgggtccgaacgcagtcctgtactacctggcagaaaacgaaagcaagctgttcgccttcatttacaaactgtttggcagcgtgccgggttgggacaaaaagttcacgacggaacagctggaggcatttaaccaccagttcgagagcagcaatttcaccgctcgtgaattggatctggagctgcaaaaggacgtggcgcgtgttattctgacccaagttggttctggcccgcaagaaacgaacgagtctctgatcgatgcgaaaaccggcctgccgaaggagtaggaaggagatataaaaatgcaaaccgaacacgtaatcctgctgaacgcacaaggcgtcccgacgggtacgctggagaaatatgcagcccacaccgctgacacccgcttgcacctggcttttagctcttggctgttcaacgcaaaaggtcaactgctggttacccgccgtgcactgagcaagaaggcgtggccgggtgtctggactaatagcgtgtgcggtcacccgcaactgggtgaaagcaatgaggacgcagtgattcgtcgttgtcgttatgaattgggtgtcgaaatcaccccgcctgaaagcatttatccggacttccgttaccgtgccaccgatccgagcggtatcgttgaaaacgaagtttgtccggtctttgcggcacgtacgaccagcgcgctgcaaatcaacgacgacgaggtgatggactaccagtggtgtgatctggccgacgttctgcatggcatcgatgccaccccgtgggccttttctccgtggatggtgatgcaggcgaccaaccgtgaggcgcgtaaacgtttgagcgcgttcacccaactgaagtaaccatgggctagaggcatcaaataaaacgaaaggctcagtcgaaagactgggcctttcgttttatctgttgtttgtcggtgaacgctctcctgagtaggacaaatccgccgccctagacctagg

SEQ ID NO: 39 - DNA, 1653 bp, Artificial Sequence
  Description of Artificial Sequence Synthetic polynucleotide
ggtaccatggaagctcgtcgctccgcgaactatgaaccgaacagctgggattatgactacctgctgtcttctgataccgacgaaagcatcgaagtgtataaagataaagccaaaaaactggaagcagaagttcgtcgcgaaatcaacaacgaaaaagcagaatttctgacgctgctggaactgatcgataacgtgcagcgtctgggcctgggttatcgcttcgaaagcgatatccgtggcgcgctggaccgctttgttagttccggcggtttcgatgctgtcaccaaaacgtcgctgcatggcaccgcgctgagctttcgtctgctgcgccagcacggtttcgaagtttctcaagaagcctttagtggcttcaaagatcagaacggtaattttctggaaaacctgaaagaagacattaaagcaatcctgtccctgtatgaagcctcatttctggcactggaaggcgaaaatattctggatgaagctaaagtcttcgcgatctctcatctgaaagagctcagtgaagaaaaaattggtaaagaactggctgaacaggttaaccatgcgctggaactgccgctgcaccgtcgcacccaacgtctggaagccgtctggtctatcgaagcatatcgcaaaaaagaagatgccaaccaggtgctgctggaactggcaattctggattataatatgatccagtccgtttaccaacgtgacctgcgcgaaacctcacgttggtggcgtcgcgtcggtctggctacgaaactgcactttgcgcgtgatcgcctgattgaatccttttattgggccgtcggtgtggcattcgaaccgcaatactcggattgccgtaatagcgtcgcgaaaatgttttcattcgtgaccattatcgatgacatttatgatgtgtacggcaccctggacgaactggaactgtttacggatgctgtggaacgctgggacgttaacgcgatcaatgatctgccggactatatgaaactgtgcttcctggccctgtataacaccattaatgaaatcgcatacgataacctgaaagacaaaggtgaaaatattctgccgtacctgacgaaagcgtgggccgatctgtgtaatgcgtttctgcaagaagcgaaatggctgtataataaaagtaccccgacgtttgatgactacttcggcaatgcctggaaatcatcgagcggtccgctgcaactggtgtttgcctacttcgcagtggttcaaaacattaaaaaagaagaaatcgaaaacctgcaaaaataccatgataccatctcgcgtccgagccacatcttccgcctgtgcaacgacctggcatctgctagtgcggaaattgcccgtggcgaaacggcaaattccgtgtcatgttatatgcgcaccaaaggtatttctgaagaactggcgacggaaagtgttatgaacctgatcgatgaaacctggaagaaaatgaacaaagaaaaactgggcggttctctgtttgcgaaaccgttcgttgaaaccgctatcaacctggcgcgtcagagtcattgtacgtaccacaatggtgatgcccatacctcgccggacgaactgacccgtaaacgtgtgctgagcgtcattaccgaaccgatcctgccgtttgaacgctaaacgcgtctgcag

SEQ ID NO: 40 - DNA, 2707 bp, Artificial Sequence
  Description of Artificial Sequence Synthetic polynucleotide
ggtaccaggaggtaaaacatatgtcgtctaaagttaaagtcgtccgccgcaccatttctactagcatccgtatgtgtcgtattaccaccgagagcggcgaaggcgttcagcgtcgtatcgcgaaccaccacagcaatctgtgggacgataacttcatccagagcctgagcacgccgtacggtgcaatctcttatcacgagagcgcgcagaaactgattggtgaggttaaagaaatgatcaacagcatcagcctgaaggacggtgagctgattaccccaagcaatgatctgctgatgcgcttgtccattgtggactccatcgaacgcttgggtatcgatcgccacttcaaaagcgagattaagtctgctttggactatgtctattcttattggaatgagaagggcatcggttggggtcgtgacagcgtggtggcagacctgaatagcaccgcactgggtctgcgcacgttgcgtctgcatggttacccagtcagcagcgacgttttgcaacacttcaaagagcagaagggtcagttcgcgtgcagcgcgattcagaccgaaggtgaaatccgttccgtcttgaacctgttccgcgctagccaaattgcgtttccgggcgaaaaggttatggaagaggcggaagtgttcagcacgatctatctgaaagaggcgatcctgaaactgccggtgtgcggcctgagccgtgagatttcgtacgttctggagtacggttggcacatcaatctgccgcgcttggaagcacgtaattacattgacgtgtttggcgaggacccgatttatctgacgccaaacatgaaaacccaaaaactgctggagctggccaagctggagttcaacatgttccactctctgcaacaacaggagctgaagctgctgagccgttggtggaaggatagcggtttcagccagatgacgtttccgcgccatcgtcacgttgagtactacacgctggctagctgtattgatagcgagccgcaacatagcagcttccgtctgggctttgcgaagatctttcacctggcaaccgtcctggatgacatttacgataccttcggtactatggacgagttggagctgttcacggcggcagtcaagcgctggcatcctagcgccaccgaatggctgccggagtatatgaaaggcgtttacatggtcctgtacgaaacggttaacgagatggcgggtgaggcggagaagagccaaggtcgcgataccttgaactacggtcgtaatgccctggaagcgtatattgatgcaagcatggaagaagctaagtggatctttagcggttttctgccgacgttcgaagaatatctggacaatggcaaagtgagctttggttacggcattggcaccctgcagccgatcctgaccctgggcattccgttcccgcatcacattctgcaggagattgactttccgagccgtctgaatgatgtcgcgtctagcattttgcgcctgaagggtgatatccatacctatcaggcggagcgctcgcgtggcgagaaaagcagctgtatctcttgctacatggaagagaatccggaatctacggaagaagatgcgatcaatcacattaacagcatggtcgacaaactgctgaaagagctgaactgggaatatctgcgtcctgacagcaatgtgccgatcaccagcaagaagcatgcgtttgatatcctgcgtgccttctatcacctgtacaaataccgtgatggtttcagcgttgccaattacgagatcaaaaatctggtcatgactaccgtgattgagccggtgccgctgtgatgaggaataaaattatgctgcgtagcctgctgcgtggcctgacccactttccgcgtgtgaactccgctcaacaaccgtcgtgcgcccatgcccgcctgcaatttcgtccgcgcagcatgcagctgctggccgaagatcgtaccgaccacatgcgtggtgctagtacgtgggcgggcggtcaatcccaagatgaactgatgctgaaagacgaatgcattctggtcgatgcggatgacaacatcaccggtcatgtgagcaaactggaatgtcataagtttctgccgcaccagccggcgggtctgctgcaccgtgcattctctgtttttctgttcgatgaccagggtcgcctgctgctgcagcaacgtgcccgcagcaagattacctttccgtctgtttggaccaatacgtgctgttcacatccgctgcacggccagaccccggatgaagtggatcagctgtcgcaagtggctgatggcacggttccgggtgcaaaagcggcggcaattcgtaagctggaacatgaactgggtatcccggcacaccagctgccggcaagtgcatttcgtttcctgacccgcctgcattattgtgctgccgatgttcagccggcggcaacccaatcagccctgtggggcgaacacgaaatggattacattctgttcatccgtgctaacgtgaccctggcgccgaatccggatgaagtggacgaagttcgttatgtcacgcaggaagaactgcgccagatgatgcaaccggataacggtctgcaatggtccccgtggtttcgcattatcgctgcgcgtttcctggaacgttggtgggccgatctggacgcagcactgaacaccgataaacacgaagactggggcaccgtccatcacatcaacgaagcataaccatgg

SEQ ID NO: 41 - protein, 6 aa, Artificial Sequence
  Description of Artificial Sequence Synthetic 6xHis tag
HHHHHH